(19) United States
(12) Patent Application Publication
(10) Pub. No.: US 2026/0097482 A1
(43) Pub. Date: Apr. 9, 2026

(54) METHOD AND APPARATUS FOR DRIVING SURGICAL INSTRUMENTS

(71) Applicant: LIVSMED INC., Seongnam-si (KR)

(72) Inventors: Young Wan KIM, Seongnam-si (KR); Yeon Taek JEONG, Suwon-si (KR); Dong Hyuk KIM, Seoul (KR)

(21) Appl. No.: 19/054,270

(22) Filed: Feb. 14, 2025

(30) Foreign Application Priority Data
Oct. 4, 2024 (KR) 10-2024-0135150

(51) Int. Cl. B25J 3/00; A61B 34/37; G05B 15/02

(52) U.S. Cl. CPC B25J 3/00; A61B 34/37; G05B 15/02

(57) ABSTRACT

Provided is a method and apparatus for driving surgical instruments. In the method, a first position reference point and a second position reference point may be set. In some embodiments, in the method, first position information regarding a relationship among the first position reference point, the second position reference point, and a position of a first robot may be generated, and second position information regarding a relationship among the first position reference point, the second position reference point, and a position of a second robot may be generated. In some embodiments, in the method, third position information regarding a relationship between the position of the first robot and the position of the second robot may be generated based on the first position information and the second position information.

### CROSS-REFERENCE TO RELATED APPLICATION

This application is based on and claims priority under 35 USC § 119 to Korean Patent Application No. 10-2024-0135150, filed on Oct. 4, 2024, in the Korean Intellectual Property Office, the disclosure of which is incorporated by reference herein in its entirety.

### BACKGROUND

### 1. Field

The present disclosure relates to a method and apparatus for driving surgical instruments.

### 2. Description of the Related Art

Medically, surgery refers to the treatment of diseases by cutting, slitting, or manipulating the skin, mucous membranes, or other tissues using medical devices. In particular, open surgery, which cuts and opens the skin of a surgical site and cures, shapes, or removes an organ therein, may cause bleeding, side effects, patient pain, scars, or the like. Accordingly, recently, surgery performed by inserting only a medical device, for example, laparoscopic surgical instrument, microsurgical microscope, and the like by forming a predetermined hole in the skin or surgery using a robot has been spotlighted as an alternative.

Here, a surgical robot refers to a robot that has a function of replacing a surgical action performed by a surgeon. Compared to humans, the surgical robot has the advantage of being able to operate with greater accuracy and precision, as well as being able to operate remotely.

Meanwhile, a surgical robot is generally composed of a master robot and a slave robot. When a surgical operator manipulates a control lever (e.g., a handle) equipped on the master robot, a surgical tool coupled to or held by a robot arm equipped on the slave robot may be manipulated to perform surgery.

In controlling a plurality of slave robots through the master robot, determining relative angles between the slave robots is an important factor. For example, determining the relative angles between the plurality of robot arms is necessary to enable the normal progression of surgery.

Accordingly, there is a need for technology capable of determining relative angles between a plurality of robotic arms in a system that utilizes the robotic arms included in a plurality of slave robots.

The aforementioned background technology is technical information possessed by the inventor for derivation of the present disclosure or acquired by the inventor during the derivation of the present disclosure, and is not necessarily prior art disclosed to the public before the application of the present disclosure.

### SUMMARY

The present disclosure is directed to providing a method and apparatus for driving surgical instruments. The present disclosure is also directed to providing a computer-readable recording medium having recorded thereon a program for executing the method on a computer.

The problem to be solved by the present disclosure is not limited to the problems mentioned above, and other problems and advantages of the present disclosure, which are not mentioned, will be understood by the following description, and will be more clearly understood by the embodiments of the present disclosure. In some embodiments, it will be appreciated that the problems and advantages to be solved by the present disclosure may be realized by means and combinations thereof indicated in the claims.

According to a first aspect of the present disclosure, there is provided a method including setting a first position reference point and a second position reference point, generating first position information regarding a relationship among the first position reference point, the second position reference point, and a position of a first robot, and generating second position information regarding a relationship among the first position reference point, the second position reference point, and a position of a second robot, and generating third position information regarding a relationship between the position of the first robot and the position of the second robot based on the first position information and the second position information.

According to a second aspect of the present disclosure, there is provided an apparatus including at least one memory, and at least one processor, wherein the at least one processor is configured to set a first position reference point and a second position reference point, generate first position information regarding a relationship among the first position reference point, the second position reference point, and a position of a first robot, and generate second position information regarding a relationship among the first position reference point, the second position reference point, and a position of a second robot, and generate third position information regarding a relationship between the position of the first robot and the position of the second robot based on the first position information and the second position information.

According to a third aspect of the present disclosure, there is provided a computer-readable recording medium having recorded thereon a program for executing the method according to the first aspect on a computer.

In some embodiments, other methods and systems for implementing the present disclosure, and a computer-readable recording medium having recorded thereon a program for executing the method may be further provided.

Other aspects, features, and advantages other than those described above will become apparent from the following drawings, claims, and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram for describing an example of a system for driving a surgical instrument according to an embodiment;

FIG. 2A is a configuration diagram illustrating an example of a user terminal according to an embodiment;

FIG. 2B is a configuration diagram illustrating an example of a server according to an embodiment;

FIG. 3 is a diagram for describing another example of the system for driving a surgical instrument according to an embodiment;

FIG. 4 is a block diagram illustrating an internal configuration of a surgical robot system of FIG. 3;

FIG. 5 is a perspective view illustrating a slave robot of the surgical robot system of FIG. 3 and a multi-joint type surgical instrument mounted on the slave robot;

FIG. 6 is a perspective view illustrating a multi-joint type surgical instrument according to an embodiment of the present disclosure;

FIGS. 7 and 8 are perspective views of an end tool of the multi-joint type surgical instrument of FIG. 6;

FIGS. 9A and 9B are plan views of the end tool of the multi-joint type surgical instrument of FIG. 6;

FIGS. 10 and 11 are perspective views of a driving part of the multi-joint type surgical instrument of FIG. 6;

FIG. 12 is a plan view of the driving part of the multi-joint type surgical instrument of FIG. 6;

FIG. 13 is a rear view of the driving part of the multi-joint type surgical instrument of FIG. 6;

FIG. 14 is a side view of the driving part of the multi-joint type surgical instrument of FIG. 6;

FIG. 15 is a view illustrating the configuration of pulleys and wires of the multi-joint type surgical instrument illustrated in FIG. 6, in detail for the configuration related to a first jaw;

FIG. 16 is a view illustrating the configuration of pulleys and wires of the multi-joint type surgical instrument illustrated in FIG. 6, in detail for the configuration related to a second jaw;

FIGS. 17A to 18C are views illustrating a pitch motion of the multi-joint type surgical instrument illustrated in FIG. 6;

FIGS. 19A to 20B are views illustrating a yaw motion of the multi-joint type surgical instrument illustrated in FIG. 6;

FIG. 21 is a flowchart for describing an example of a method of driving a surgical instrument according to an embodiment;

FIGS. 22A and 22B are views for describing an example of a first robot and a second robot according to an embodiment;

FIG. 23 is a view for describing an example of generating first position information and second position information using positions of the first robot and the second robot, respectively, illustrated in FIGS. 22A and 22B;

FIG. 24 is a diagram for describing an example of generating third position information by a processor according to an embodiment; and

FIG. 25 is a view for describing an example of a surgical instrument operating based on driving information according to an embodiment.

### DETAILED DESCRIPTION

Hereinafter, various embodiments of the present disclosure are described with reference to the accompanying drawings. While the present disclosure is susceptible to various modifications and may have several embodiments, specific embodiments thereof are shown by way of example in the drawings and will herein be described in detail. However, it should be understood that there is no intent to limit the present disclosure to the specific embodiments, but on the contrary, the present disclosure is to cover all modifications, equivalents, and alternatives falling within the spirit and scope of the present disclosure. With regard to description of the drawings, like reference numerals have been used for like components.

Expressions such as “includes” or “may include” that may be used in various embodiments of the present disclosure indicate the existence of a corresponding function, operation, or component that is disclosed, and are not intended to limit one or more additional functions, operations, or components. In some embodiments, in the various embodiments of the present disclosure, it is to be understood that the terms such as “including,” “having,” and the like are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

In various embodiments of the present disclosure, the expression “or” includes any and all combinations of one or more of the associated listed items. For example, “A or B” may include “A,” “B,” or “both A and B.” In some embodiments, in the present disclosure, the expressions “A or B,” “at least one of A or B,” “one or more of A and/or B,” and the like may include all possible combinations of the listed items. For example, “A or B,” “at least one of A and B,” or “at least one of A or B” may refer to all of the following cases: (1) including at least one A, (2) including at least one B, or (3) including at least one A and at least one B.

While expressions such as “first” and “second” used in the various embodiments of the present disclosure may describe various components of the various embodiments, the corresponding components are not limited by the expressions such as “first” and “second.” For example, these expressions do not limit the order and/or importance of corresponding components. These expressions may be used to distinguish one component from another. For example, both a first user device and a second user device are user devices and indicate different user devices. For example, a first component may be named a second component or a second component may be named a first component without departing from the scope of the various embodiments of present disclosure.

In an embodiment of the present disclosure, the terms “module,” “unit,” “part,” or the like are terms which designate a component that performs at least one function or operation, and the component may be implemented with a hardware or software, or a combination of hardware and software. In some embodiments, a plurality of “modules,” a plurality of “units,” or a plurality of “parts,” except for “a module,” “a unit,” or a “part” which needs to be implemented to a specific hardware, may be integrated to at least one module or a chip and implemented in at least one processor.

The expression “configured to” used in the present disclosure may be interchangeably used with other expressions such as “suitable for,” “having the capacity to,” “designed to,” “adapted to,” “made to,” or “capable of,” depending on cases. The term “configured to” may not necessarily mean that a device is “In some embodiments designed to” in terms of hardware.

The terms used in various embodiments of the present disclosure are used to describe a particular embodiment only and are not intended to limit the various embodiments of the present disclosure. Singular forms are intended to include plural forms as well, unless the context clearly indicates otherwise.

Unless otherwise defined, all terms used herein, including technical or scientific terms, have the same meanings as those generally understood by those with ordinary knowledge in the field of art to which the various embodiments of the present disclosure belongs.

Generally used terms defined in a dictionary should be interpreted to have meanings the same as meanings in the context of the related art and are not interpreted as ideal or excessively formal meanings unless the various embodiments of the present disclosure clearly define otherwise.

Hereinafter, various embodiments of the present disclosure will be described in detail with reference to the accompanying drawings.

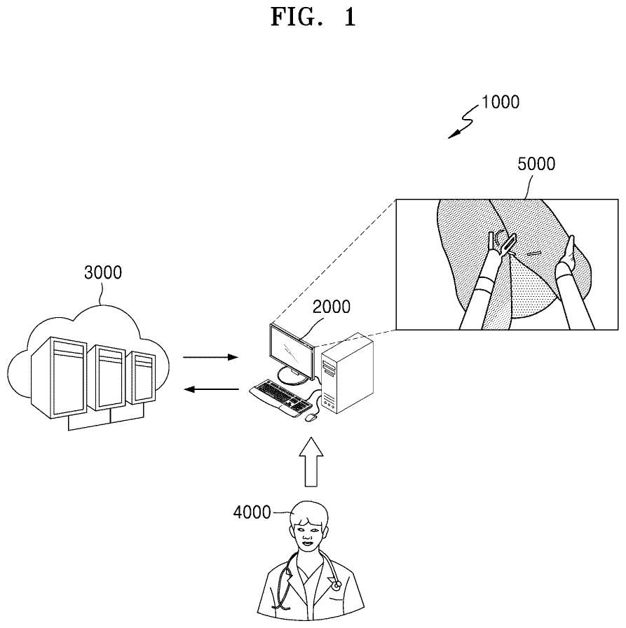

FIG. 1 is a diagram for describing an example of a system for driving a surgical instrument according to an embodiment.

Referring to FIG. 1, a system 1000 includes a user terminal 2000 and a server 3000. For example, the user terminal 2000 and the server 3000 may be connected to each other by a wired or wireless communication method to transmit and receive data (e.g., a first position reference point, a second position reference point, first position information, second position information, third position information, driving information, or the like) to and from each other.

For convenience of description, in FIG. 1, the system 1000 is illustrated as including the user terminal 2000 and the server 3000, but the present disclosure is not limited thereto. For example, the system 1000 may include another external device (not shown), and operations of the user terminal 2000 and the server 3000 to be described below may be implemented by a single device (e.g., the user terminal 2000 or the server 3000) or a plurality of devices.

The user terminal 2000 may include a display device and a device (e.g., a keyboard, a mouse, or the like) for receiving an input of a user 4000, and may be a computing device including a memory and a processor. For example, the display device may be implemented as a touch screen and may receive user input. For example, the user terminal 2000 may correspond to a notebook personal computer (PC), a desktop PC, a laptop computer, a tablet computer, a smartphone, and the like, but the present disclosure is not limited thereto.

The server 3000 may be a device that communicates with an external device (not shown) including the user terminal 2000. As an example, the server 3000 may be a device that stores various pieces of data including manipulation information regarding the motions of the user 4000, the first position reference point, the second position reference point, the first position information, the second position information, the third position information, and the like.

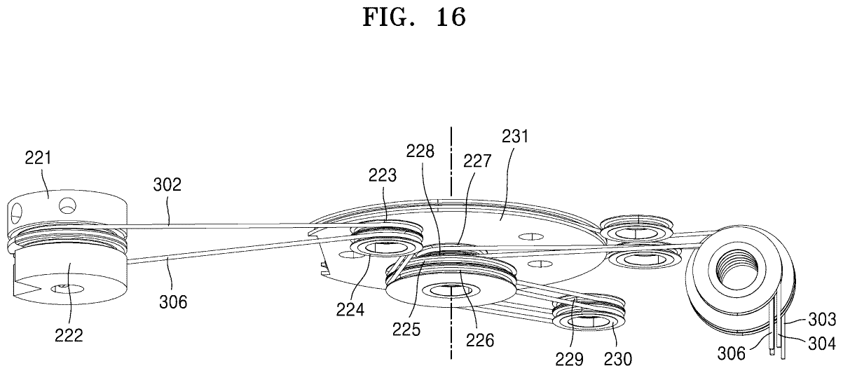

Alternatively, the server 3000 may be a computing device that includes memory and a processor and has its own computing capabilities. As an example, the server 3000 may perform at least some of operations of the user terminal 2000 to be described later with reference to FIGS. 1 to 25. For example, the server 3000 may be a cloud server, but the present disclosure is not limited thereto.

Based on a position of a first robot and a position of a second robot, the user terminal 2000 may generate third position information regarding a relationship between the position of the first robot and the position of the second robot For example, the user terminal 2000 may calculate a relative angle between the first robot and the second robot by taking into consideration of the position of the first robot and the position of the second robot, and generate the third position information including the relative angle between the first and second robots.

For example, the user terminal 2000 may set a first position reference point and a second position reference point. In some embodiments, the user terminal 2000 may generate first position information regarding a relationship among the first position reference point, the second position reference point, and the position of the first robot. In some embodiments, the user terminal 2000 may generate second position information regarding a relationship among the first position reference point, the second position reference point, and the position of the second robot. In some embodiments, the user terminal 2000 may generate the third position information regarding the relationship between the position of the first robot and the position of the second robot based on the first position information and the second position information.

The first position information may include relative position information of the first robot, which is determined based on the first position reference point and the second position reference point. For example, the first position information may include a vector value for the position of the first robot determined based on the first position reference point and the second position reference point. In some embodiments, the first position information may include a vector value calculated as a difference between a vector value determined based on the position of the first robot and the first position reference point, and a vector value determined based on the position of the first robot and the second position reference point.

The second position information may include relative position information of the second robot, which is determined based on the first position reference point and the second position reference point. For example, the second position information may include a vector value for the position of the second robot determined based on the first position reference point and the second position reference point. In some embodiments, the second position information may include a vector value calculated as a difference between a vector value determined based on the position of the second robot and the first position reference point, and a vector value determined based on the position of the second robot and the second position reference point.

The first position reference point may refer to a point at which a first-1 component of the first robot and a second-1 component of the second robot are located. The first-1 component may refer to a component included as part of a first robot arm of the first robot. The second-1 component may refer to a component included as part of a first robot arm of the second robot, and may be a component corresponding to the first-1 component of the first robot.

The second position reference point may refer to a point at which a first-2 component of the first robot and a second-2 component of the second robot are located. The first-2 component may refer to a component included as part of a second robot arm of the first robot. The second-2 component may refer to a component included as part of a second robot arm of the second robot, and may be a component corresponding to the first-2 component of the second robot.

In other words, some components of each of the first and second robots may be located at each of the first position reference point and the second position reference point. Some components of the first and second robots may be located simultaneously at both the first and second position reference points, but may also be located at different times, and the present disclosure is not limited to the examples described above.

Meanwhile, the user terminal 2000 may calculate a first vector value for the first position reference point and the second position reference point based on the position of the first robot. For example, the user terminal 2000 may calculate a first-1 intermediate vector value based on the first position reference point and the position of the first robot. In some embodiments, the user terminal 2000 may calculate a first-2 intermediate vector value based on the second position reference point and the position of the first robot. In some embodiments, the user terminal 2000 may calculate the first vector value based on the first-1 intermediate vector value and the first-2 intermediate vector value. Here, the first position information may include the first vector value, and the second position information may include a second vector value.

In some embodiments, the user terminal 2000 may calculate the second vector value for the first position reference point and the second position reference point based on the position of the second robot. For example, the user terminal 2000 may calculate a second-1 intermediate vector value based on the first position reference point and the position of the second robot. In some embodiments, the user terminal 2000 may calculate a second-2 intermediate vector value based on the second position reference point and the position of the second robot. In some embodiments, the user terminal 2000 may calculate the second vector value based on the second-1 intermediate vector value and the second-2 intermediate vector value.

Meanwhile, the user terminal 2000 may generate the third position information regarding the relationship between the position of the first robot and the position of the second robot based on a correlation between the first position information and the second position information. Here, the third position information may include a relative angle generated based on the position of the first robot and the position of the second robot.

In some embodiments, the user terminal 2000 may control at least one of the first robot and the second robot based on the third position information.

For example, the user terminal 2000 may generate driving information based on operations of the master robot controlling the first robot and the second robot, and on a transformation relationship among the position of the first robot equipped with a camera, the position of the second robot equipped with a surgical instrument, and a position of the surgical instrument. In some embodiments, the user terminal 2000 may control at least one of the first and second robots based on the driving information.

In some embodiments, the user terminal 2000 may generate first intermediate driving information based on a transformation relationship between a position of an image captured by the camera and the position of the first robot and operation information of the master robot. In some embodiments, the user terminal 2000 may generate second intermediate driving information based on a transformation relationship between the position of the first robot and the position of the second robot, and the first intermediate driving information. In some embodiments, the user terminal 2000 may generate the driving information based on the transformation relationship between the position of the surgical instrument and the position of the second robot, and the second intermediate driving information.

As an example, when generating second driving information, the user terminal 2000 may use the transformation relationship between the position of the first robot and the position of the second robot. In some embodiments, the user terminal 2000 may generate the second driving information using information regarding a transformation matrix from a coordinate system of the second robot, which is equipped with the surgical instrument, to a coordinate system of the first robot, which is equipped with the camera.

Meanwhile, the user terminal 2000 may generate the third position information regarding the relationship between the position of the first robot and the position of the second robot or drive at least one of the first robot and the second robot, through an application installed in the user terminal 2000. Here, the application may be a software program installed for the purpose of driving a surgical instrument (e.g., the surgical instrument mounted on the second robot) of the user 4000. For example, through the application, the user 4000 may perform various medical activities, such as generating the first, second, and third position information or controlling the surgical instrument based on the third position information.

Meanwhile, the user terminal 2000 may output an image 5000 indicating the motion of the surgical instrument driven based on the motion of the user 4000. For example, the user terminal 2000 may control the motion of the surgical instrument based on the third position information, and the controlled motion of the surgical instrument may be output through the image 5000 and verified by the user 4000. The image 5000, representing a motion of the surgical instrument, allows the user 4000 to intuitively understand the motion of the surgical instrument in relation to the motion of the user 4000, and more accurately manipulate the surgical instrument.

Meanwhile, for convenience of description, it has been described throughout the specification that the user terminal 2000 sets the first position reference point and the second position reference point, generates the first position information regarding the relationship among the first position reference point, the second position reference point, and the position of the first robot, generates the second position information regarding the relationship among the first position reference point, the second position reference point, and the position of the second robot, and generates the third position information regarding the relationship between the position of the first robot and the position of the second robot based on the first position information and the second position information, but the present disclosure is not limited thereto. For example, at least some of operations performed by the user terminal 2000 may be performed by the server 3000.

In other words, at least some of operations of the user terminal 2000 to be described with reference to FIGS. 1 to 25 may be performed by the server 3000. For example, the server 3000 may set the first position reference point and the second position reference point. In some embodiments, the server 3000 may generate the first position information regarding the relationship among the first position reference point, the second position reference point, and the position of the first robot. In some embodiments, the server 3000 may generate the second position information regarding the relationship among the first position reference point, the second position reference point, and the position of the second robot. In some embodiments, the server 3000 may generate the third position information regarding the relationship between the position of the first robot and the position of the second robot based on the first position information and the second position information.

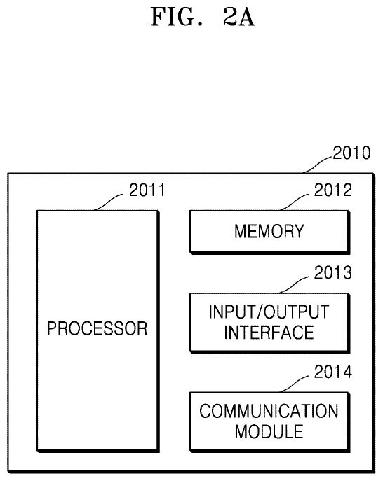

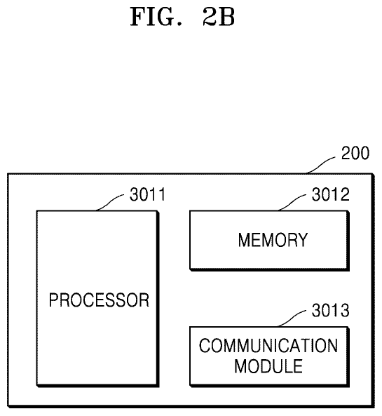

FIG. 2A is a configuration diagram illustrating an example of a user terminal according to an embodiment.

Referring to FIG. 2A, a user terminal 2010 includes a processor 2011, a memory 2012, an input/output interface 2013, and a communication module 2014. For convenience of description, only components related to the present disclosure are illustrated in FIG. 2A. Accordingly, other general-purpose components In some embodiments to the components illustrated in FIG. 2A may be further included in the user terminal 2010. In some embodiments, it will be apparent to those skilled in the art related to the present disclosure that the processor 2011, the memory 2012, the input/output interface 2013, and the communication module 2014 illustrated in FIG. 2A may be implemented as independent devices.

The processor 2011 may process instructions of a computer program by performing a basic arithmetic operation, a logic operation, and an input/output operation. Here, the instructions may be provided from the memory 2012 or an external device (e.g., the server 3000 or the like). In some embodiments, the processor 2011 may control overall operations of the other components included in the user terminal 2010.

The processor 2011 may set the first position reference point and the second position reference point. The component of the first robot and the component of the second robot may move and be located at each of the first position reference point and the second position reference point. For example, the first-1 component of the first robot and the second-1 component of the second robot may be located at the first position reference point, and the first-2 component of the first robot and the second-2 component of the second robot may be located at the second position reference point. Each of the first-1 component and the first-2 component of the first robot may be a component included in the robot arm of the first robot. Each of the second-1 component and the second-2 component of the second robot may be a component included in the robot arm of the second robot.

In some embodiments, the processor 2011 may generate the first position information regarding the relationship among the first position reference point, the second position reference point, and the position of the first robot. In some embodiments, the processor 2011 may generate the second position information regarding the relationship among the first position reference point, the second position reference point, and the position of the second robot.

The first position information may include the relative position information of the first robot, which is determined based on the first position reference point and the second position reference point. The second position information may include the relative position information of the second robot, which is determined based on the first position reference point and the second position reference point.

In some embodiments, the processor 2011 may generate the third position information regarding the relationship between the position of the first robot and the position of the second robot based on the first position information and the second position information.

Meanwhile, the processor 2011 may calculate the first vector value for the first position reference point and the second position reference point based on the position of the first robot. In some embodiments, the processor 2011 may calculate the second vector value for the first position reference point and the second position reference point based on the position of the second robot. Here, the first vector value may be included in the first position information, and the second vector value may be included in the second position information.

In some embodiments, the processor 2011 may calculate the first-1 intermediate vector value based on the first position reference point and the position of the first robot, calculate the first-2 intermediate vector value based on the second position reference point and the position of the first robot, and calculate the first vector value based on the first-1 intermediate vector value and the first-2 intermediate vector value. In some embodiments, the processor 2011 may calculate the second-1 intermediate vector value based on the first position reference point and the position of the second robot, calculate the second-2 intermediate vector value based on the second position reference point and the position of the second robot, and calculate the second vector value based on the second-1 intermediate vector value and the second-2 intermediate vector value.

Meanwhile, the processor 2011 may generate the third position information regarding the relationship between the position of the first robot and the position of the second robot based on the correlation between the first position information and the second position information. Here, the third position information may include the relative angle generated based on the position of the first robot and the position of the second robot.

Meanwhile, the processor 2011 may control at least one of the first robot and the second robot based on the third position information. For example, the processor 2011 may generate driving information based on the operations of the master robot controlling the first robot and the second robot, and on the transformation relationship among the position of the first robot equipped with the camera, the position of the second robot equipped with the surgical instrument, and the position of the surgical instrument. In some embodiments, the processor 2011 may control at least one of the first robot and the second robot based on the driving information.

In some embodiments, the processor 2011 may generate the first intermediate driving information based on the transformation relationship between the position of the image captured by the camera and the position of the first robot and the operation information of the master robot. In some embodiments, the processor 2011 may generate the second intermediate driving information based on the transformation relationship between the position of the first robot and the position of the second robot and the first intermediate driving information. In some embodiments, the processor 2011 may generate the driving information based on the transformation relationship between the position of the surgical instrument and the position of the second robot and the second intermediate driving information. In some embodiments, the processor 2011 may control at least one of the first robot and the second robot based on the driving information.

Meanwhile, the processor 2011 may generate the operation information of the master robot based on a member that allows the position and function of the surgical instrument to be manipulated by the user's motion. The operation information of the master robot may include the manipulation information regarding the user's motion. For example, the processor 2011 may generate the manipulation information regarding the user's motion based on a member that allows the position and function of the surgical instrument to be manipulated by the user's motion.

The member that allows the position and function of the surgical instrument to be manipulated by the user's motion may be a member provided in the form of a handle-shaped manipulation member, but is not limited thereto, and may be implemented in many different forms to achieve the same purpose. For example, a portion of the member may be provided in the form of a handle, and the other portions thereof may be provided in different forms, such as a clutch button. In some embodiments, a finger insertion tube may be further formed so as to allow the surgical operator's finger to be inserted therethrough and fixed to facilitate manipulation of the surgical tool.

Meanwhile, the member for manipulating the position and function of the surgical instrument by the user's motion may be a component included in the master robot. For example, the processor 2011 may generate the operation information of the master robot based on the member that allows the position and function of the surgical instrument to be manipulated by the user's motion.

The manipulation information refers to information indicating a user's intuitive motion to manipulate the position and function of the surgical instrument. In some embodiments, the manipulation information may include position information and orientation information, in a physical coordinate system, of the member that allows a user to manipulate the position and function of the surgical instrument.

Meanwhile, the processor 2011 may generate manipulation information based on position information and orientation information of a member that allows a user to manipulate the position and function of the surgical instrument. For example, the processor 2011 may generate the manipulation information using a difference between initial position and orientation information of the member that allows a user to manipulate the position and function of the surgical instrument, and position and orientation information of the member after the user's motion.

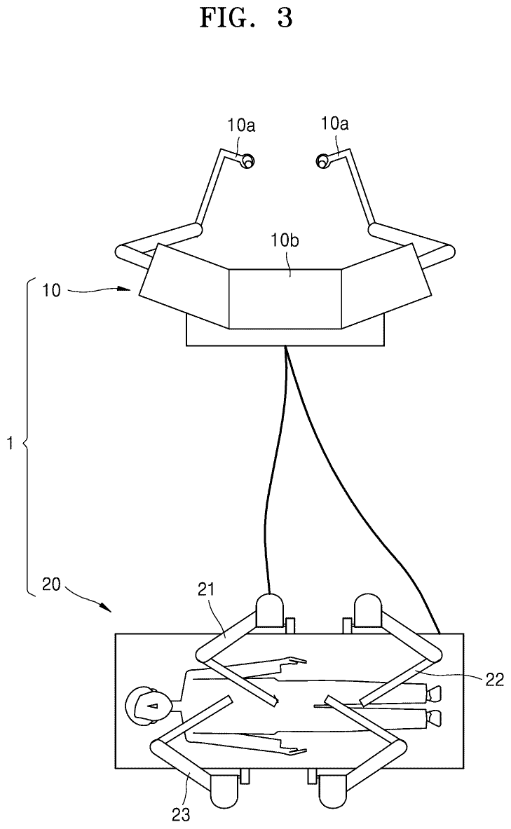

Specific examples in which the processor 2011 according to an embodiment operates will be described with reference to FIGS. 3 to 25.

The processor 2011 may be implemented in an array of multiple logic gates, or in a combination of a universal microprocessor and a memory that stores a program executable in the microprocessor. For example, the processor 2011 may include a general-purpose processor, a central processing unit (CPU), a microprocessor, a digital signal processor (DSP), a controller, a microcontroller, a state machine, or the like. In some environments, the processor 2011 may include an application-specific semiconductor (ASIC), a programmable logic device (PLD), a field programmable gate array (FPGA), or the like. For example, the processor 2011 may refer to a combination of processing devices such as, for example, a combination of a DSP and a microprocessor, a combination of a plurality of microprocessors, a combination of one or more microprocessors in conjunction with a DSP core, or a combination of any other such configuration.

The memory 2012 may include any non-transitory computer-readable recording medium. As an example, the memory 2012 may include a permanent mass storage device such as a random access memory (RAM), a read-only memory (ROM), a disk drive, a solid state drive (SSD), a flash memory, or the like. In another example, the permanent mass storage device such as a ROM, SSD, a flash memory, a disk drive, or the like may be a separate permanent storage device which is distinguishable from the memory. In some embodiments, an operating system OS and at least one program code e.g., a code for the processor 2011 to perform operations to be described later with reference to FIGS. 3 to 25 may be stored in the memory 2012.

These software components may be loaded from a computer-readable recording medium separate from the memory 2012. The separate computer-readable recording medium may be a recording medium that may be directly connected to the user terminal 2010, and may include, for example, a computer-readable recording medium, such as a floppy drive, a disk, a tape, a DVD/CD-ROM drive, a memory card, or the like. Alternatively, the software components may be loaded into the memory 2012 through the communication module 2014 instead of the computer-readable recording medium. For example, at least one program may be loaded into the memory 2012 based on a computer program (for example, a computer program for performing, by the processor 2011, operations to be described later with reference to FIGS. 3 to 25) installed by the files provided through the communication module 2014 by developers or a computer file distribution system that distributes the installation files of applications.

The input/output interface 2013 may be a member for an interface with a device (e.g., a keyboard, a mouse, or the like) for input or output, the member being connected to the user terminal 2010 or being included in the user terminal 2010. The input/output interface 2013 may be configured separately from the processor 2011, but the present disclosure is not limited thereto, and the input/output interface 2013 may be configured to be included in the processor 2011.

The communication module 2014 may provide a configuration or a function for the server 3000 and the user terminal 2010 to communicate with each other through a network. In some embodiments, the communication module 2014 may provide a configuration or function for the user terminal 2010 to communicate with another external device. For example, a control signal, a command, data, or the like, which is provided according to the control of the processor 2011, may be transmitted to the server 3000 and/or an external device through the communication module 2014 and the network.

Meanwhile, although not shown in FIG. 2A, the user terminal 2010 may further include a display device. For example, the display device may be implemented as a touch screen. Alternatively, the user terminal 2010 may be connected to an independent display device through a wired or wireless communication method to transmit/receive data to or from each other. For example, a video, an image, or the like of driving the surgical instrument may be provided through the display device by using driving information.

FIG. 2B is a configuration diagram illustrating an example of the server according to an embodiment.

Referring to FIG. 2B, a server 3010 includes a processor 3011, a memory 3012, and a communication module 3013. For convenience of description, only components related to the present disclosure are illustrated in FIG. 2B. Accordingly, other general-purpose components other than the components illustrated in FIG. 2B may be further included in the server 3010. In some embodiments, it will be apparent to those skilled in the art related to the present disclosure that the processor 3011, the memory 3012, and the communication module 3013 illustrated in FIG. 2B may be implemented as independent devices.

The processor 3011 may set the first position reference point and the second position reference point. In some embodiments, the processor 3011 may generate the first position information regarding the relationship among the first position reference point, the second position reference point, and the position of the first robot, and generate the second position information regarding the relationship among the first position reference point, the second position reference point, and the position of the second robot. In some embodiments, the processor 3011 may generate the third position information regarding the relationship between the position of the first robot and the position of the second robot based on the first position information and the second position information.

In other words, at least one of the operations of the processor 2011 described above with reference to FIG. 2A may be performed by the processor 3011. In this case, the user terminal 2010 may output information transmitted from the server 3010 through the display device.

Meanwhile, an implementation example of the processor 3011 is the same as the implementation example of the processor 2011 described above with reference to FIG. 2A, and thus a detailed description thereof will be omitted.

Various data such as data required for an operation of the processor 3011 and data generated according to the operation of the processor 3011 may be stored in the memory 3012. In some embodiments, an operating system (OS) and at least one program (e.g., a program necessary for the operation of the processor 3011, or the like) may be stored in the memory 3012.

Meanwhile, an implementation example of the memory 3012 is the same as the implementation example of the memory 2012 described above with reference to FIG. 2A, and thus a detailed description thereof will be omitted.

The communication module 3013 may provide a configuration or function for the server 3010 and the user terminal 2010 to communicate with each other through a network. In some embodiments, the communication module 2014 may provide a configuration or function for the server 3010 to communicate with another external device. For example, a control signal, a command, data, or the like, which is provided according to the control of the processor 3011, may be transmitted to the user terminal 2010 and/or an external device through the communication module 3013 and the network.

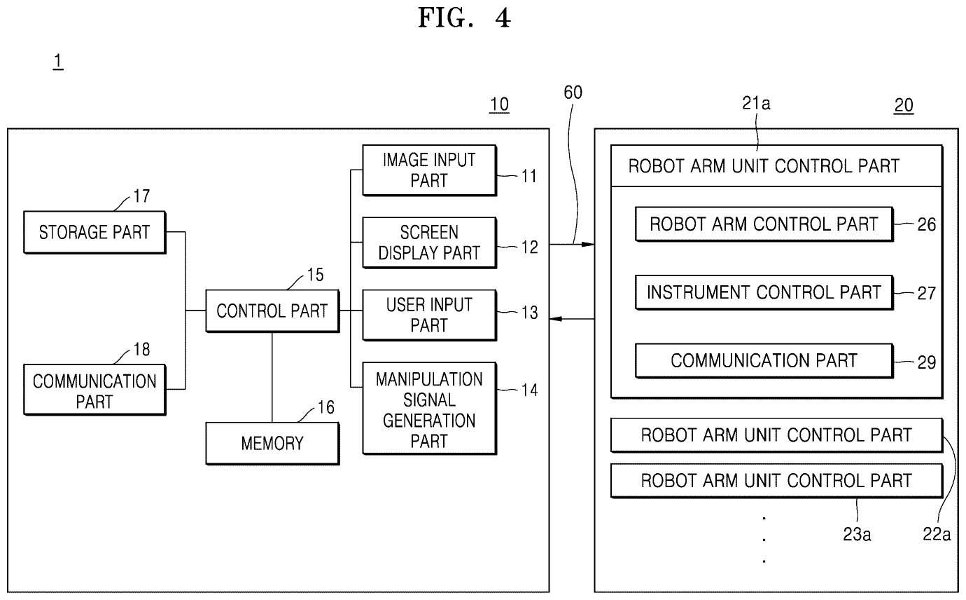

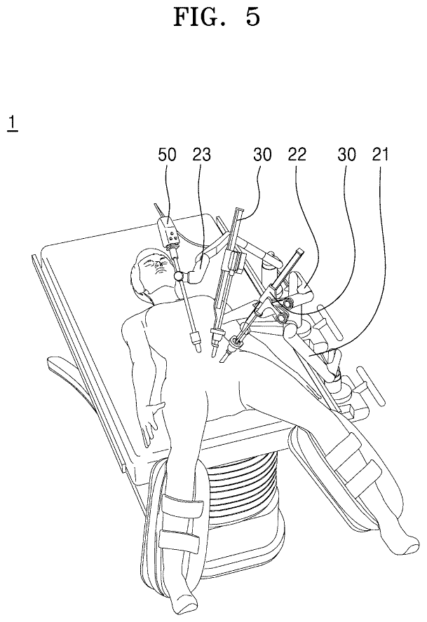

FIG. 3 is a diagram for describing another example of the system for driving a surgical instrument according to an embodiment, FIG. 4 is a block diagram illustrating an internal configuration of the surgical robot system of FIG. 3, and FIG. 5 is a perspective view illustrating a slave robot of the surgical robot system of FIG. 3 and a multi-joint type surgical instrument mounted on the slave robot.

Referring to FIGS. 3 to 5, a surgical robot system 1 includes a master robot 10, a slave robot 20, and a multi-joint type surgical instrument 30.

The master robot 10 includes manipulating members 10a and a display member 10b, and the slave robot 20 includes one or more robot arm units 21, 22, and 23.

The master robot 10 includes the manipulating members 10a so that a surgical operator can grip and manipulate them respectively with both hands. The manipulating members 10a may be implemented as two or more handles as illustrated in FIG. 3, and manipulation signals according to the handle manipulation of the surgical operator are transmitted to the slave robot 20 through a wired or wireless communication network so that the robot arm units 21, 22, and 23 are controlled. That is, surgical motions such as positioning, rotation, and cutting operations of the robot arm units 21, 22, and 23 may be performed by the handle manipulation of the surgical operator. Here, the manipulation signals may include one or more of manipulation information regarding a user's motion, and first driving information or second driving information regarding a motion of the surgical instrument, which are described above.

For example, the surgical operator may manipulate the robot arm units 21, 22, and 23 using manipulation levers in the form of a handle. The manipulation lever as described above may have various mechanical configurations according to the manipulate method thereof, and may be provided in various configurations for operating the robot arm units 21, 22, and 23 of the slave robot 20 and/or other surgical instruments, such as a master handle manipulating the motion of each of the robot arm units 21, 22, and 23 and various input tools added to the master robot 10 for manipulating the functions of the entire system such as joystick, keypad, trackball, foot pedal, and touch screen. Here, the manipulating member 10a is not limited to the shape of a handle and can be applied without any limitation as long as it can control motions of the robot arm units 21, 22, and 23 through a network such as a wired or wireless communication network.

Meanwhile, according to an embodiment of the present disclosure, the manipulation information may be generated based on the manipulation lever or the manipulating member 10a described above. For example, according to an embodiment of the present disclosure, the manipulation information may be generated based on a motion of a user who manipulates the manipulation lever or the manipulating member 10a. However, examples of generating the manipulation information are not limited to the above description.

Alternatively, a voice input, a motion input, or the like may also be applied to the surgical robot system 1 for user input. That is, a user may wear, on the head thereof, glasses or a head mount display (HMD), to which a sensor is attached, and a laparoscope 50 may move according to a direction in which the user's gaze. Alternatively, when the user issues a command with voice, such as “left,” “right,” “first arm,” “second arm,” and the like, the voice command may be recognized and the motion may be performed. For example, in the present disclosure, according to an embodiment, manipulation information may be generated based on a user's voice, first driving information may be calculated based on the manipulation information, the presence of a risk associated with a motion of the surgical instrument may be determined based on the first driving information, and the surgical instrument may be driven based on the result of determining the presence of the risk.

An image captured through the laparoscope 50 to be described later is displayed as a screen image on the display member 10b of the master robot 10. In some embodiments, a predetermined virtual manipulation plate may be displayed independently or displayed together with the image captured by the laparoscope 50 on the display member 10b.

The display member 10b may include one or more monitors, each of which may individually display information necessary for surgery. The quantity of monitors may be variously determined depending on the type or kind of information that needs to be displayed.

Meanwhile, the slave robot 20 may include one or more robot arm units 21, 22, and 23. Here, each of the robot arm units 21, 22, and 23 may be provided in the form of a module that can operate independently of each other, and in this case, an algorithm for preventing a collision between the robot arm units 21, 22, and 23 may be applied to the surgical robot system 1.

In general, a robot arm refers to a device having a function similar to that of the arm and/or the wrist of a human being and having a wrist portion to which a predetermined tool may be attached. In the present disclosure, the robot arm units 21, 22, and 23 may each be defined as a concept encompassing all of the components such as an upper arm, a lower arm, a wrist, and an elbow, a multi-joint type surgical instrument coupled to the wrist portion, and the like. Alternatively, the robot arm unit may also be defined as a concept that includes only components for driving the multi-joint type surgical instrument, excluding the multi-joint type surgical instrument coupled to the wrist portion.

The robot arm units 21, 22, and 23 of the slave robot 20 described above may be implemented to be driven with multiple degrees of freedom. The robot arm units 21, 22, and 23 may include, for example, a surgical instrument inserted into a surgical site of a patient, a yaw driving part for rotating the surgical instrument in a yaw direction according to a surgical position, a pitch driving part for rotating the surgical instrument in a pitch direction perpendicular to a rotational driving of the yaw driving part, a transfer driving part for moving the surgical instrument in a length direction, a rotation driving part for rotating the surgical instrument, and a surgical instrument driving part for incising or cutting the surgical lesion by driving an end effector at an end of the surgical instrument. However, the configuration of the robot arm units 21, 22, and 23 is not limited thereto, and it should be understood that this example does not limit the scope of the present disclosure. Here, a detailed description of the actual control process, such as rotation and movement of the robot arm units 21, 22, and 23 in a corresponding direction by the surgical operator manipulating the manipulating member 10a will be omitted.

Here, two of the robot arm units 21, 22, and 23 may each have the multi-joint type surgical instrument 30 attached thereto, and one of the robot arm units 21, 22, and 23 may have the laparoscope 50 attached thereto. In some embodiments, the surgical operator may select the robot arm unit 21, 22, or 23 to be controlled via the master robot 10. As described above, by directly controlling a total of three or more surgical instruments through the master robot 10, the surgical operator may accurately and freely control various tools according to the intention of the surgical operator without a surgical assistant.

Meanwhile, one or more slave robots 20 may be provided to operate the patient, and the laparoscope 50 for allowing a surgical site to be displayed as a screen image through the display member 10b may be implemented as an independent slave robot 20. In some embodiments, as described above, the embodiments of the present disclosure can be used universally for surgeries in which various surgical endoscopes other than laparoscopes (e.g., thoracoscopes, arthroscopes, rhinoscopes, and the like) are used.

Meanwhile, the master robot 10 may generate manipulation information regarding a user's motion for driving the surgical instrument, calculate first driving information based on the manipulation information, determine the presence of a risk associated with a motion of the surgical instrument based on the first driving information, and drive the surgical instrument based on the result of determining the presence of the risk. In some embodiments, the master robot 10 may update the first driving information based on the result of determining the presence of the risk.

For example, the master robot 10 may transmit the first driving information to the slave robot 20 via a wired or wireless communication network to control the robot arm units 21, 22, and 23. That is, surgical motions such as positioning, rotation, and cutting operations of the robot arm units 21, 22, and 23 may be performed by the handle manipulation of the surgical operator.

Referring to FIG. 4, in an embodiment of the present disclosure, the master robot 10 may include an image input part 11, a screen display part 12, a user input part 13, a manipulation signal generation part 14, a control part 15, a memory 16, a storage part 17, and a communication part 18.

Meanwhile, the master robot 10 may be included in the user terminal of FIG. 2A. For example, the manipulation signal generation part 14, the control part 15, and the like are included in the processor 2011, the memory 16, the storage part 17, and the like are included in the memory 2012, and the communication part 18 may be included in the communication module 2014, but the example of the master robot 10 is not limited to the above description.

The image input part 11 may receive an image captured by a camera provided in the laparoscope 50 of the slave robot 20 through a wired or wireless communication network. The image captured by the camera may include an image representing the motion of the surgical instrument driven by using the first driving information or the second driving information.

The screen display part 12 outputs a screen image corresponding to the image received through the image input part 11 as visual information. In some embodiments, the screen display part 12 may further output information corresponding to biometric information of a subject to be treated, when the biometric information is input. In some embodiments, the screen display part 12 may further output image data (e.g., an X-ray image, a computerized tomography (CT) image, a magnetic resonance imaging (MRI) image, or the like) associated with a patient for a surgical site. Here, the screen display part 12 may be implemented in the form of a display member (see 10b of FIG. 3), and an image processing process for allowing the received image to be output as a screen image through the screen display part 12 may be performed by the control part 15. Here, the image may include an image representing the motion of the surgical instrument driven by using the first driving information or the second driving information.

In the embodiment illustrated in FIG. 4, the image input part and the screen display part are illustrated as being included in the master robot 10, but the present disclosure is not limited thereto. The display member may be provided as a separate member spaced apart from the master robot 10. Alternatively, the display member may be provided as one component of the master robot 10. In some embodiments, in another embodiment, a plurality of display members may be provided, one of which may be disposed adjacent to the master robot 10, and the others thereof may be disposed at some distance from the master robot 10.

Here, the screen display part 12 (that is, the display member 10b of FIG. 3) may be provided as a three-dimensional display device. In some embodiments, the three-dimensional display device refers to an image display device in which depth information is added to a two-dimensional image by applying a stereoscopic technique, and this depth information is used to enable an observer to feel a three-dimensional living feeling and a sense of reality. The surgical robot system 1 according to an embodiment of the present disclosure may provide a more realistic virtual environment to a user by including a three-dimensional display device as the screen display part 12.

The user input part 13 is a member for allowing the surgical operator to manipulate the positions and functions of the robot arm units 21, 22, and 23 of the slave robot 20. The user input part 13 may be provided in the form of a handle-shaped manipulation member (see 10a of FIG. 3) as illustrated in FIG. 3, but the shape thereof is not limited thereto and may be implemented by being modified in various shapes to achieve the same purpose. In some embodiments, for example, a portion of the user input part 13 may be provided in the form of a handle, and the other portions thereof may be provided in different forms, such as a clutch button. In some embodiments, a finger insertion tube or insertion ring may be further formed so as to allow the surgical operator's finger to be inserted therethrough and fixed to facilitate manipulation of the surgical tool.

Meanwhile, according to an embodiment of the present disclosure, the manipulation information may be generated based on a motion of the surgical operator with respect to the user input part 13. For example, according to an embodiment of the present disclosure, the manipulation information may be generated based on a motion of the surgical operator who manipulates the user input part 13. However, examples of generating the manipulation information are not limited to the above description.

When the surgical operator manipulates the user input part 13 to control positional movements or surgical motions of the robot arm units 21, 22, and 23, the manipulation signal generation part 14 may generate a manipulation signal corresponding thereto. As an example, when the surgical operator manipulates the user input part 13 to control the positional movements or surgical motions of the robot arm units 21, 22, and 23, the manipulation signal generation part 14 may generate manipulation information corresponding thereto.

For example, the manipulation signal generation part 14 transmits the generated manipulation signal to the control part 15 or transmits the generated manipulation signal to the slave robot 20 through the communication part 18. The manipulation signal may be transmitted and received via a wired or wireless communication network. Based on the transmitted manipulation signal, the control part 15 may control the slave robot 20 or the multi-joint type surgical instrument 30 to operate. Alternatively, based on the transmitted manipulation signal, the robot arm control part 26 included in the slave robot 20 may control the robot arm units 21, 22, and 23 to operate. Alternatively, based on the transmitted manipulation signal, the instrument control part 27 included in the slave robot 20 may control the multi-joint type surgical instrument 30 to operate However, the method by which the motion of the slave robot 20 or the multi-joint type surgical instrument 30 is controlled based on the manipulation signal is not limited to the above description.

The instrument control part 27 may receive a manipulation signal generated by the manipulation signal generation part 14 of the master robot 10, and may serve to control the multi-joint type surgical instrument 30 so as to operate according to the manipulation signal.

The control part 15 is a kind of central processing device, and controls operations of each component so that the above-described functions can be performed. As an example, the control part 15 may perform a function of converting an image input through the image input part 11 into a screen image to be displayed through the screen display part 12. In another example, the control part 15 may generate first driving information regarding positional movements or surgical motions of the robot arm units 21, 22, and 23 based on the manipulation information. In some embodiments, the control part 15 may determine the presence of a risk associated with the positional movements or the surgical motions of the robot arm units 21, 22, and 23 based on the first driving information. In some embodiments, the control part 15 may update the first driving information based on the result of determining the presence of the risk. In some embodiments, the control part 15 may drive the robot arm units 21, 22, and 23 based on the result of determining the presence of the risk. In some embodiments, the control part 15 may calculate second driving information based on the first driving information, and may drive the robot arm units 21, 22, and 23 based on the second driving information.

Meanwhile, according to the above description, it has been described that the control part 15 calculates the first driving information, updates the first driving information, or calculates the second driving information based on the first driving information, but the present disclosure is not limited thereto, and other control parts (e.g., the robot arm control part 26, the instrument control part 27, and the like) according to the present disclosure may perform those operations.

The memory 16 may perform a function of temporarily or permanently storing data processed by the control part 15. Here, the memory 16 may include a magnetic storage medium or a flash storage medium, but the scope of the present disclosure is not limited thereto.

The storage part 17 may store data received from the slave robot 20. In some embodiments, the storage part 17 may store various pieces of input data (e.g., patient data, device data, surgery data, and the like).

The communication part 18 interworks with a communication network 60 to provide a communication interface necessary for transmitting and receiving image data transmitted from the slave robot 20 and control data transmitted from the master robot 10. The image data transmitted from the slave robot 20 may include an image representing a motion of the surgical instrument driven by using the first driving information or the second driving information. The control data transmitted from the master robot 10 may include first driving information or second driving information regarding a motion of the slave robot 20.

The slave robot 20 includes a plurality of robot arm unit control parts 21a, 22a, and 23a. In some embodiments, the robot arm unit control part 21a includes the robot arm control part 26, the instrument control part 27, and a communication part 29. In some embodiments, the robot arm unit control part 21a may further include a rail control part 28.

The robot arm control part 26 may receive a manipulation signal generated by the manipulation signal generation part 14 of the master robot 10, and may serve to control the robot arm units 21, 22, and 23 so as to operate according to the manipulation signal. For example, the robot arm control part 26 may serve to receive first driving information or second driving information calculated (or updated) by the master robot 10, and control the robot arm units 21, 22, and 23 to operate according to the first driving information or the second driving information.

The instrument control part 27 may receive a manipulation signal generated by the manipulation signal generation part 14 of the master robot 10, and may serve to control the multi-joint type surgical instrument 30 so as to operate according to the manipulation signal. For example, the instrument control part 27 may serve to receive first driving information calculated (or updated) by the master robot 10 and control the multi-joint type surgical instrument 30 to operate according to the first driving information.

The communication part 29 interworks with the communication network 60 to provide a communication interface necessary for transmitting and receiving image data transmitted from the slave robot 20 and control data transmitted from the master robot 10. The image data transmitted from the slave robot 20 may include an image representing a motion of the surgical instrument driven by using the first driving information or the second driving information. The control data transmitted from the master robot 10 may include first driving information or second driving information regarding a motion of the slave robot 20.

Meanwhile, the communication network 60 serves to connect the master robot 10 to the slave robot 20. That is, the communication network 60 refers to a communication network for providing an access path so that data can be transmitted and received between the master robot 10 and the slave robot 20 after the master robot 10 and the slave robot 20 are connected. The communication network 60 may be, for example, a wired network such as local area networks (LANs), wired area networks (WANs), metropolitan area networks (MANs), and integrated service digital networks (ISDNs), or a wireless network such as wireless LANs, code division multiple access (CDMA), Bluetooth, and satellite communication, but the scope of the present disclosure is not limited thereto.

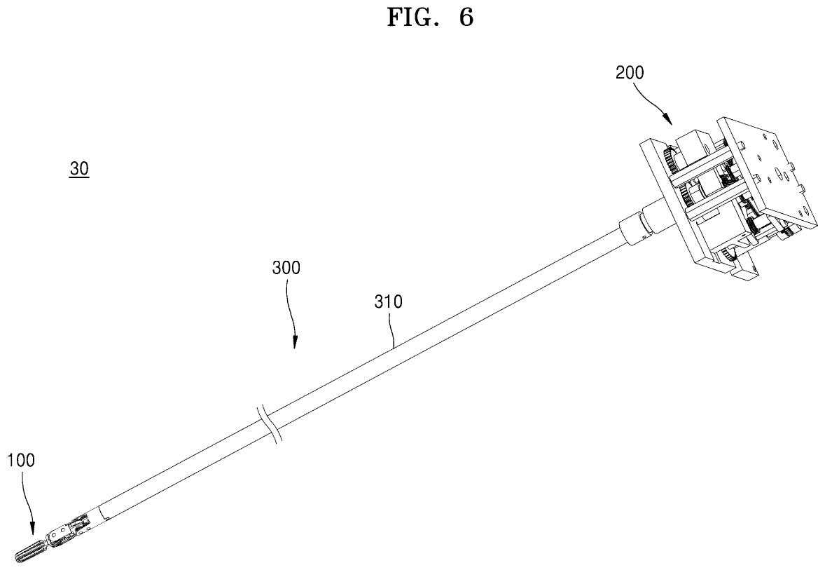

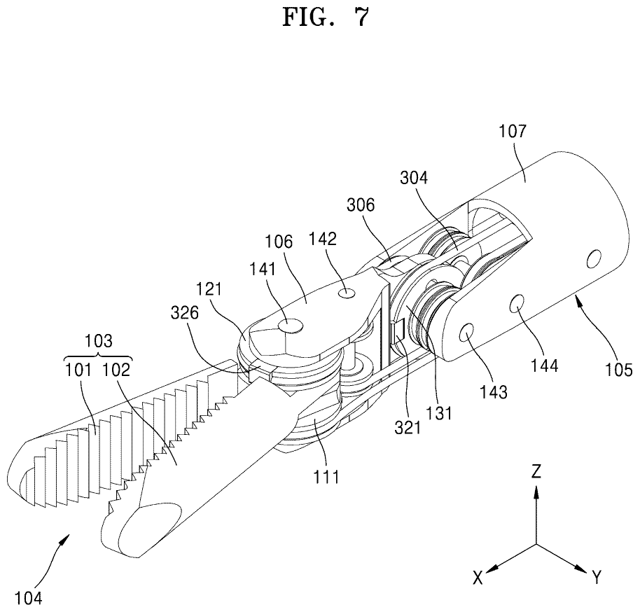

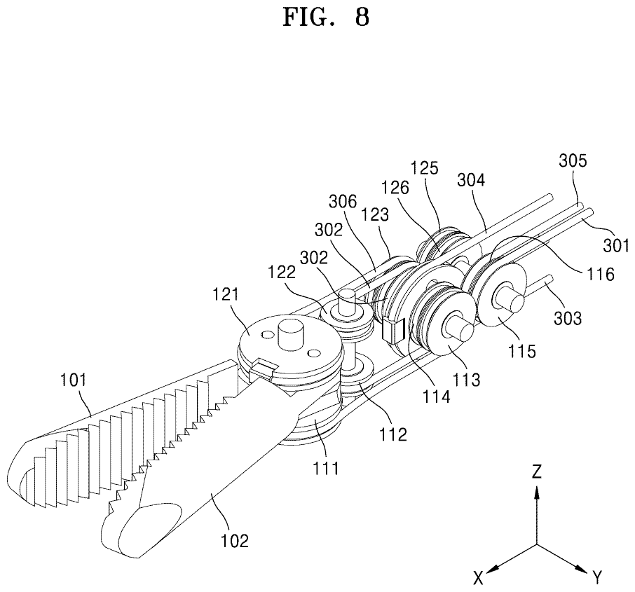

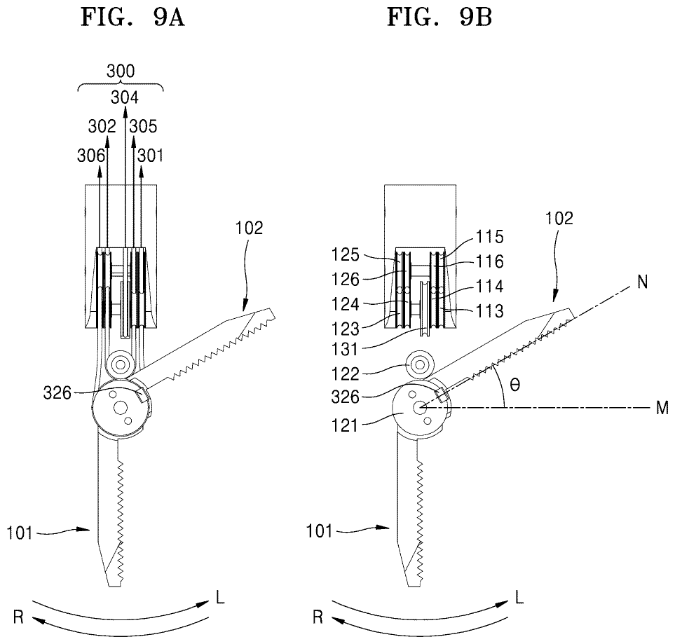

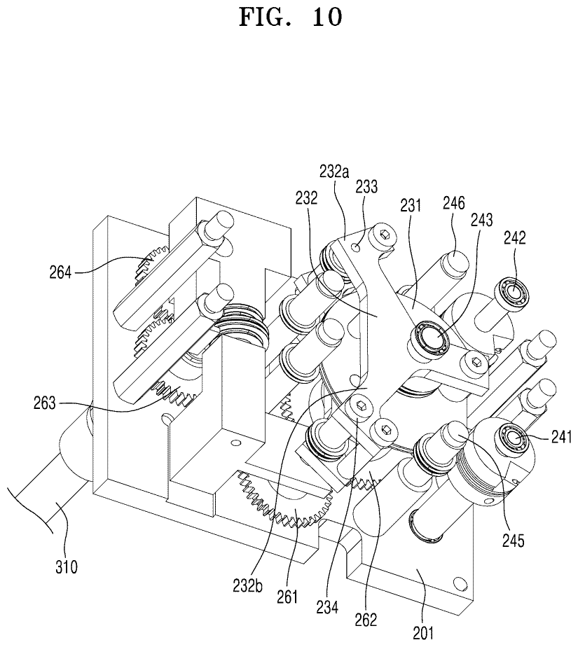

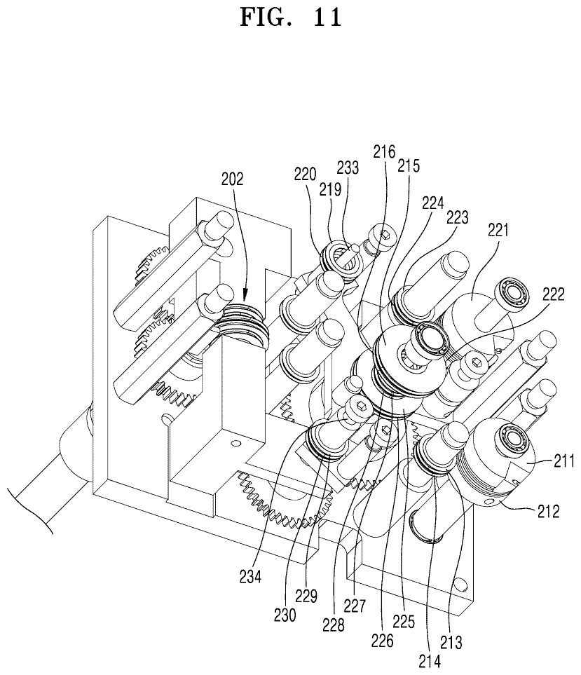

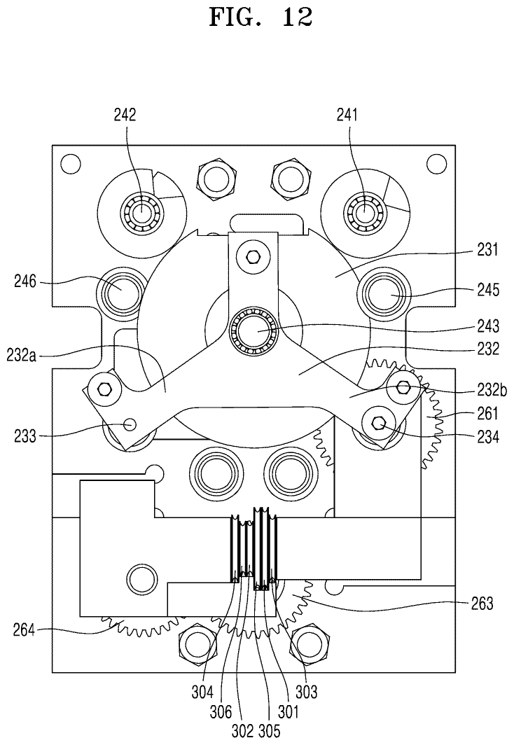

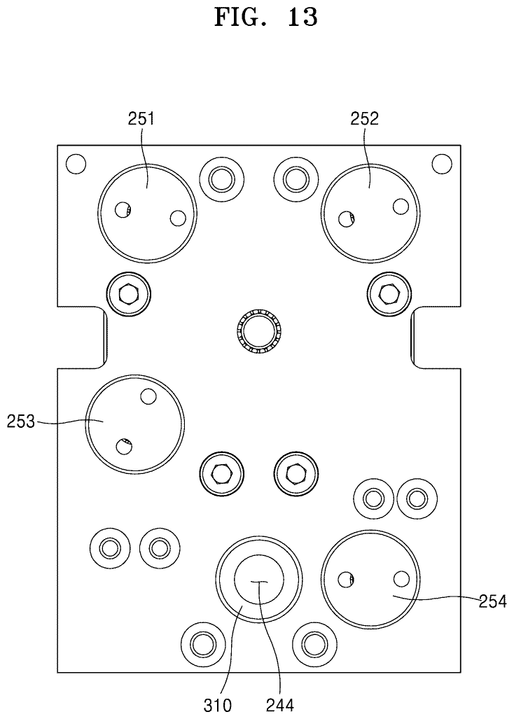

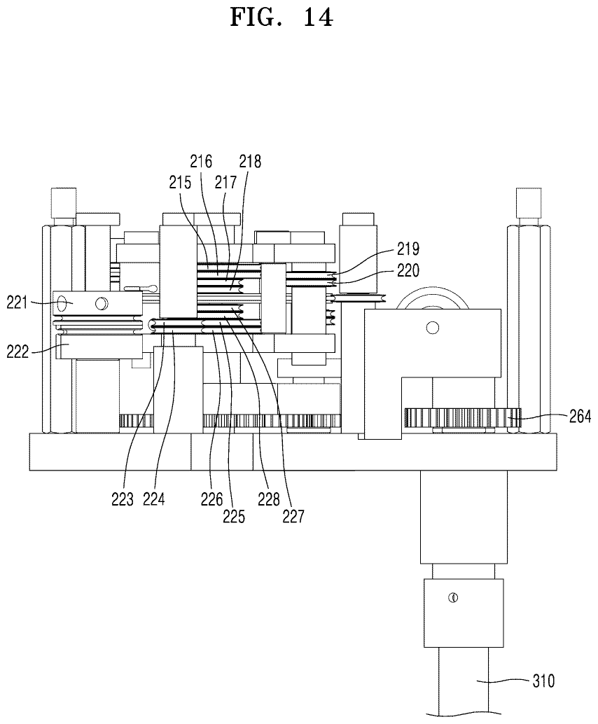

FIG. 6 is a perspective view illustrating a multi-joint type surgical instrument according to an embodiment of the present disclosure, FIGS. 7 and 8 are perspective views of an end tool of the multi-joint type surgical instrument of FIG. 6, and FIG. 9 is a plan view of the end tool of the multi-joint type surgical instrument of FIG. 6. FIGS. 10 and 11 are perspective views of a driving part of the multi-joint type surgical instrument of FIG. 6, FIG. 12 is a plan view of the driving part of the multi-joint type surgical instrument of FIG. 6, FIG. 13 is a rear view of the driving part of the multi-joint type surgical instrument of FIG. 6, and FIG. 14 is a side view of the driving part of the multi-joint type surgical instrument of FIG. 6.

Referring first to FIG. 6, the multi-joint type surgical instrument 30 according to an embodiment of the present disclosure may include an end tool 100, a driving part 200, and a power transmission part 300, and the power transmission part 300 may include a connection part 310.

The connection part 310 is formed in the shape of a hollow shaft, in which one or more wires (to be described later) may be accommodated, and may have one end portion to which the driving part 200 is coupled and another end portion to which the end tool 100 is coupled and serve to connect the driving part 200 to the end tool 100.

The driving part 200 is formed at one end portion of the connection part 310 and provides an interface capable of being coupled to the robot arm unit (see 21 or the like in FIG. 3). Accordingly, when a user operates the master robot (see 10 in FIG. 3), a motor (not shown) of the robot arm unit (see 21 or the like in FIG. 3) is operated so that the end tool 100 of the multi-joint type surgical instrument 30 can perform a motion corresponding thereto, and a driving force of the motor (not shown) is transmitted to the end tool 100 through the driving part 200. Viewed from another perspective, it may be described that the driving part 200 itself becomes an interface that connects between the multi-joint type surgical instrument 30 and the slave robot 20.

For example, when the user input part 13 (see FIG. 3) is operated by a user, a motor (not shown) of the robot arm unit 21 or the like (see FIG. 3) operates so that the end tool 100 of the multi-joint type surgical instrument 30 can perform a motion corresponding thereto, and a driving force of the motor (not shown) may be transmitted to the end tool 100 through the driving part 200.

The end tool 100 is formed on another end portion of the connection part 310, and performs necessary motions for surgery by being inserted into a surgical site. In an example of the above-described end tool 100, as shown in FIG. 7, a pair of jaws 101 and 102 for performing a grip motion may be used. However, the embodiment of the present disclosure is not limited thereto, and various devices for performing surgery may be used as the end tool 100. For example, a configuration such as a cantilever cautery may also be used as the end tool. The above-described end tool 100 is connected to the driving part 200 by the power transmission part 300 and receives a driving force through the power transmission part 300 to perform a motion necessary for surgery, such as a gripping motion, a cutting motion, a suturing motion, or the like.

Here, the end tool 100 of the multi-joint type surgical instrument 30 according to an embodiment of the present disclosure is formed to be rotatable in at least two or more directions, for example, the end tool 100 may be formed to perform a pitch motion around a rotation shaft 143 of FIG. 7 and simultaneously perform a yaw motion and an actuation motion around a rotation shaft 141 of FIG. 7.

Here, each of a pitch motion, a yaw motion, an actuation motion, and a roll motion as used in the present disclosure are defined as follows.

First, the pitch motion means a motion of the end tool 100 rotating in a vertical direction with respect to an extension direction of the connection part 310 (an X-axis direction of FIG. 6), that is, a motion rotating around the Y-axis of FIG. 6. In other words, the pitch motion means a motion of the end tool 100, which is formed to extend from the connection part 310 in the extension direction of the connection part 310 (the X-axis direction of FIG. 6), rotating vertically around the Y-axis with respect to the connection part 310.

Next, the yaw motion means a motion of the end tool 100 rotating in left and right directions, that is, a motion rotating around a Z-axis of FIG. 6, with respect to the extension direction of the connection part 310 (the X-axis direction of FIG. 6). In other words, the yaw motion means a motion of the end tool 100, which is formed to extend from the connection part 310 in the extension direction of the connection part 310 (the X-axis direction of FIG. 6), rotating horizontally around the Z-axis with respect to the connection part 310. That is, the yaw motion means a motion of two jaws 101 and 102, which are formed on the end tool 100, rotating around the Z-axis in the same direction.

Meanwhile, the actuation motion means a motion of the end tool 100 rotating around the same shaft of rotation as that of the yaw motion, while the two jaws 101 and 102 rotate in the opposite directions so as to be closed or opened. That is, the actuation motion means rotating motions of the two jaws 101 and 102, which are formed on the end tool 100, in the opposite directions around the Z-axis.

Defining this from another perspective, the yaw rotation may be defined as a motion in which an end tool jaw pulley to be described later rotates around the rotation shaft 141, which is an end tool jaw pulley rotation shaft, and the pitch rotation may be defined as a motion in which the end tool jaw pulley revolves around the rotation shaft 143, which is an end tool pitch rotation shaft.

The roll motion refers to a motion in which the multi-joint type surgical instrument rotates with the connection part 310 as a shaft. For example, the roll motion may be a motion in which the multi-joint type surgical instrument rotates in the clockwise or counterclockwise direction around the extension direction of the connection part 310 (the X-axis direction of FIG. 6).

Meanwhile, the roll motion may mean a motion in which the end tool 100 rotates around the X-axis with respect to the connection part 310. For example, the roll motion may be a motion in which the end tool rotates in the clockwise or counterclockwise direction around the extension direction of the connection part 310 (the X-axis direction of FIG. 7).

The power transmission part 300 may connect the driving part 200 to the end tool 100, transmit the driving force from the driving part 200 to the end tool 100, and include a plurality of wires, pulleys, links, sections, gears, or the like. Hereinafter, the end tool 100, the driving part 200, the power transmission part 300, and the like of the multi-joint type surgical instrument 30 of FIG. 6 will be described in more detail.

Hereinafter, the power transmission part 300 of the multi-joint type surgical instrument 30 of FIG. 6 will be described in more detail.

Referring to FIGS. 6 to 14, the power transmission part 300 of the multi-joint type surgical instrument 30 according to an embodiment of the present disclosure may include a wire 301, a wire 302, a wire 303, a wire 304, a wire 305, and a wire 306.

Here, the wires 301 and 305 may be paired to serve as first jaw wires. The wires 302 and 306 may be paired to serve as second jaw wires. Here, the components encompassing the wires 301 and 305, which are first jaw wires, and the wires 302 and 306, which are second jaw wires, may be referred to as jaw wires. In some embodiments, the wires 303 and 304 may be paired to serve as pitch wires.

Here, in the drawings, a pair of wires are illustrated as being associated with a rotational motion of a first jaw 101, and a pair of wires are illustrated as being associated with a rotational motion of a second jaw 102, but an embodiment of the present disclosure is not limited thereto. For example, a pair of wires may be associated with a yaw motion, and a pair of wires may be associated with an actuation motion.

In some embodiments, the power transmission part 300 of the multi-joint type surgical instrument 30 according to an embodiment of the present disclosure may include a coupling member 321, a coupling member 326, and the like, which are coupled to respective end portions of the wires in order to couple the wires to the pulleys. Here, each of the coupling members may have various shapes as necessary, such as a ball shape, a tube shape, and the like.

Here, the coupling member 321, which is a pitch wire coupling member, is coupled to the end portions of the wires 303 and 304, which are pitch wires, at the end tool 100 side to serve as a pitch wire-end tool coupling member. Meanwhile, although not illustrated in the drawings, a pitch wire-driving part coupling member (not shown) may be coupled to the end portions of the wires 303 and 304, which are pitch wires, at the driving part 200 side.

Meanwhile, the coupling member 326, which is a second jaw wire coupling member, is coupled to the end portions of the wires 302 and 306, which are second jaw wires, at the end tool 100 side to serve as a second jaw wire-end tool coupling member. Meanwhile, although not illustrated in the drawings, a second jaw wire-driving part coupling member (not shown) may be coupled to the end portions of the wires 302 and 306, which are second jaw wires, at the driving part 200 side.

Meanwhile, although not illustrated in the drawings, a coupling member (not shown) having the same shape as the coupling member 326 may be coupled to the end portions of the wires 301 and 305, which are first jaw wires, at the end tool 100 side to serve as a first jaw wire-end tool coupling member. Meanwhile, although not illustrated in the drawings, a first jaw wire-driving part coupling member (not shown) may be coupled to the end portions of the wires 301 and 305, which are first jaw wires, at the driving part 200 side.

Here, each of the coupling members is classified as being included in the power transmission part 300, but the coupling members may be classified such that the coupling member at the end tool 100 side may be included in the end tool 100, and the coupling member at the driving part 200 side may be included in the driving part 200.

The coupling relationship between the wires, the fastening members, and the respective pulley will be described in detail as follows.

First, the wires 302 and 306, which are second jaw wires, may be a single wire. The coupling member 326, which is a first jaw wire-end tool coupling member, is inserted at an intermediate point of the second jaw wire, which is a single wire, and the coupling member 326 is crimped and fixed, and then, both strands of the second jaw wire centered on the coupling member 326 may be referred to as the wire 302 and the wire 306, respectively.

Alternatively, the wires 302 and 306, which are second jaw wires, may also be formed as separate wires, and connected to each other by the coupling member 326.

In some embodiments, by coupling the coupling member 326 to a pulley 121, the wires 302 and 306 may be fixedly coupled to the pulley 121. This allows the pulley 121 to rotate as the wires 302 and 306 are pulled and released.

Meanwhile, the second jaw wire-driving part coupling member (not shown) may be coupled to the end portions of the wires 302 and 306, which are opposite to the end portions to which the coupling member 326 is coupled. That is, the second jaw wire-driving part coupling member (not shown) may be fixed to each of the wires 302 and 306 by inserting the opposite end portions of the wires 302 and 306 into the second jaw wire-driving part coupling member (not shown) and crimping the coupling member (not shown).

In some embodiments, by coupling the second jaw wire-driving part coupling member (not shown) coupled to the wires 302 and 306 to each of the pulley 221 and the pulley 222, the wire 302 and the wire 306 may be fixedly coupled to the pulley 221 and the pulley 222, respectively. As a result, when the pulley 221 and the pulley 222 are rotated by a motor or a human force, the pulley 121 of the end tool 100 may be rotated as the wire 302 and the wire 306 are pulled and released.

Here, a driving part second jaw pulley may include two pulleys of the pulley 221 and the pulley 222, and thus the second jaw wire-driving part coupling member may also include two coupling members. Alternatively, the driving part second jaw pulley includes one pulley, the second jaw wire-driving part coupling member also includes one coupling member, and the wires 302 and 306 may be coupled to one coupling member to be coupled to one driving part second jaw pulley.

In the same manner, the wire 301 and the wire 305, which are first jaw wires, are coupled to the first jaw wire-end tool coupling member (not shown) and the first jaw wire-driving part coupling member (not shown), respectively. In some embodiments, the first jaw wire-end tool coupling member (not shown) is coupled to a pulley 111, and the first jaw wire-driving part coupling member (not shown) is coupled to a pulley 211 and a pulley 212. As a result, when the pulleys 211 and 212 are rotated by a motor or a human force, the pulley 111 of the end tool 100 may be rotated as the wire 301 and the wire 305 are pulled and released.

In the same manner, each of one end portions of the wires 303 and 304, which are pitch wires, is coupled to the coupling member 321, which is a pitch wire-end tool coupling member, and another end portion of each of the wires 303 and 304 are coupled to the pitch wire-driving part coupling member (not shown). In some embodiments, the coupling member 321 is coupled to a pulley 131, and the pitch wire-driving part coupling member (not shown) is coupled to a pulley 231. As a result, when the pulley 231 is rotated by a motor or a human force, the pulley 131 of the end tool 100 may be rotated as the wire 303 and the wire 304 are pulled and released.

As a result, the wire 301 and the wire 305, which are both strands of the first jaw wire, are coupled to a coupling member 323, which is a first jaw wire-end tool coupling member, and the first jaw wire-driving part coupling member (not shown) so as to form as a whole a closed loop. Similarly, the second jaw wire and the pitch wire may each be formed to form a closed loop.

Hereinafter, the end tool 100 of the multi-joint type surgical instrument 30 of FIG. 6 will be described in more detail.

FIGS. 7 and 8 are perspective views illustrating the end tool of the multi-joint type surgical instrument of FIG. 6, and FIG. 7 is a plan view illustrating the end tool of the multi-joint type surgical instrument of FIG. 6. Here, FIG. 7 illustrates a state in which an end tool hub 106 and a pitch hub 107 are coupled, and FIG. 8 illustrates a state in which the end tool hub 106 and the pitch hub 107 are removed.

Referring to FIGS. 7 to 9, the end tool 100 according to an embodiment of the present disclosure includes a pair of jaws for performing a grip motion, that is, the first jaw 101 and the second jaw 102. Here, each of the first jaw 101 and the second jaw 102, or a component encompassing the first jaw 101 and the second jaw 102 may be referred to as a jaw 103.

In some embodiments, the end tool 100 may include the pulley 111, a pulley 112, a pulley 113, a pulley 114, a pulley 115, and a pulley 116 that are related to a rotational motion of the first jaw 101. In some embodiments, the end tool 100 may include the pulley 121, a pulley 122, a pulley 123, a pulley 124, a pulley 125, and a pulley 126 that are related to a rotational motion of the second jaw 102.

Here, in the drawings, one group of pulleys are illustrated as being associated with a rotational motion of the first jaw 101, and one group of pulleys are illustrated as being associated with a rotational motion of the second jaw 102, but an embodiment of the present disclosure is not limited thereto. For example, one group of pulleys in the end tool may be associated with a yaw motion, and one group of pulleys in the end tool may be associated with an actuation motion. Here, the pulleys included in the end tool 100, including the pulleys described above, may be collectively referred to as end tool pulleys.

Meanwhile, the pulleys facing each other are illustrated in the drawings as being formed parallel to each other, but an embodiment of the present disclosure is not limited thereto, and each of the pulleys may be variously formed with a position and a size suitable for the configuration of the end tool.

In some embodiments, the end tool 100 according to an embodiment of the present disclosure may include the end tool hub 106 and the pitch hub 107.

The rotation shaft 141 and a rotation shaft 142, which will be described later, may be inserted through the end tool hub 106, and the end tool hub 106 may internally accommodate at least some of the first jaw 101 and the second jaw 102, which are axially coupled to the rotation shaft 141. In some embodiments, the end tool hub 106 may internally accommodate at least some of the pulley 112 and the pulley 122 that are axially coupled to the rotation shaft 142.

In some embodiments, the pulley 131 serving as an end tool pitch pulley may be formed at one end portion of the end tool hub 106. As shown in FIG. 7, the pulley 131 may be formed as a separate member from the end tool hub 106 and coupled to the end tool hub 106. Alternatively, although not illustrated in the drawings, the pulley 131 may be integrally formed with the end tool hub 106 as one body. That is, one end portion of the end tool hub 106 is formed in a disk shape or a semi-circular shape such as a pulley, and a groove around which a wire can be wound may be formed on an outer circumferential surface thereof. The wires 303 and 304 described above are coupled to the pulley 131 serving as an end tool pitch pulley, and a pitch motion may be performed as the pulley 131 is rotated around the rotation shaft 143.

The rotation shaft 143 and a rotation shaft 144, which will be described later, may be inserted through the pitch hub 107, and the pitch hub 107 may be axially coupled to the end tool hub 106 and the pulley 131 by the rotation shaft 143. Thus, the end tool hub 106 and the pulley 131 (coupled thereto) may be formed to be rotatable around the rotation shaft 143 with respect to the pitch hub 107.

In some embodiments, the pitch hub 107 may internally accommodate at least some of the pulley 113, the pulley 114, the pulley 123, and the pulley 124 that are axially coupled to the rotation shaft 143. In some embodiments, the pitch hub 107 may internally accommodate at least some of the pulley 115, the pulley 116, the pulley 125, and the pulley 126 that are axially coupled to the rotation shaft 144.

In some embodiments, the end tool 100 according to an embodiment of the present disclosure may include the rotation shaft 141, the rotation shaft 142, the rotation shaft 143, and the rotation shaft 144. As described above, the rotation shaft 141 and the rotation shaft 142 may be inserted through the end tool hub 106, and the rotation shaft 143 and the rotation shaft 144 may be inserted through the pitch hub 107.

The rotation shaft 141, the rotation shaft 142, the rotation shaft 143, and the rotation shaft 144 may be arranged sequentially from a distal end 104 of the end tool 100 toward a proximal end 105 thereof. Accordingly, starting from the distal end 104, the rotation shaft 141 may be referred to as a first pin, the rotation shaft 142 may be referred to as a second pin, the rotation shaft 143 may be referred to as a third pin, and the rotation shaft 144 may be referred to as a fourth pin.

Here, the rotation shaft 141 may function as an end tool jaw pulley rotation shaft, the rotation shaft 142 may function as an end tool jaw auxiliary pulley rotation shaft, the rotation shaft 143 may function as an end tool pitch rotation shaft, and the rotation shaft 144 may function as an end tool pitch auxiliary rotation shaft of the end tool 100.

Each of the rotation shafts 141, 142, 143, and 144 may be fitted into one or more pulleys, which will be described in detail below.

The pulley 111 functions as an end tool first jaw pulley, and the pulley 121 functions as an end tool second jaw pulley, and these two components may be collectively referred to as end tool jaw pulleys.

The pulley 111 and the pulley 121, which are end tool jaw pulleys, are formed to face each other, and are formed to be rotatable independently of each other around the rotation shaft 141, which is an end tool jaw pulley rotation shaft. Here, in the drawings, it is illustrated that the pulley 111 and the pulley 121 are formed to rotate around one rotation shaft 141, but it is of course possible that each end tool jaw pulley may be formed to be rotatable around a separate shaft. Here, the first jaw 101 may be fixedly coupled to the pulley 111 and rotated together with the pulley 111, and the second jaw 102 may be fixedly coupled to the pulley 121 and rotated together with the pulley 121. Yaw and actuation motions of the end tool 100 are performed according to the rotation of the pulley 111 and the pulley 121. That is, when the pulley 111 and the pulley 121 are rotated in the same direction around the rotation shaft 141, the yaw motion is performed, and when the pulley 111 and the pulley 121 are rotated in opposite directions around the rotation shaft 141, the actuation motion is performed.

Here, the first jaw 101 and the pulley 111 may be formed as separate members and coupled to each other, or the first jaw 101 and the pulley 111 may be integrally formed as one body. Similarly, the second jaw 102 and the pulley 121 may be formed as separate members and coupled to each other, or the second jaw 102 and the pulley 121 may be integrally formed as one body.

The pulley 112 functions as an end tool first jaw auxiliary pulley, and the pulley 122 functions as an end tool second jaw auxiliary pulley, and these two components may be collectively referred to as end tool jaw auxiliary pulleys.

In some embodiments, the pulley 112 and the pulley 122, which are end tool jaw auxiliary pulleys, may be additionally provided on one side of the pulley 111 and one side of the pulley 121, respectively. In other words, the pulley 112, which is an auxiliary pulley, may be disposed between the pulley 111 and the pulley 113/pulley 114. In some embodiments, the pulley 122, which is an auxiliary pulley, may be disposed between the pulley 121 and the pulley 123/pulley 124. The pulley 112 and the pulley 122 may be formed to be rotatable independently of each other around the rotation shaft 142. Here, in the drawings, it is illustrated that the pulley 112 and the pulley 122 are formed to rotate around one rotation shaft 142, but it is of course possible that each of the pulley 112 and the pulley 122 may be formed to be rotatable around a separate shaft. Such auxiliary pulleys will be described in more detail later.

The pulley 113 and the pulley 114 function as end tool first jaw pitch main pulleys, and the pulley 123 and the pulley 124 function as end tool second jaw pitch main pulleys, and these two components may be collectively referred to as end tool jaw pitch main pulleys.

The pulley 115 and the pulley 116 function as end tool first jaw pitch sub-pulleys, and the pulley 125 and the pulley 126 function as end tool second jaw pitch sub-pulleys, and these two components may be collectively referred to as end tool jaw pitch sub-pulleys.

Hereinafter, components related to the rotation of the pulley 111 will be described.

The pulley 113 and the pulley 114 function as end tool first jaw pitch main pulleys. That is, the pulley 113 and the pulley 114 function as main rotation pulleys for a pitch motion of the first jaw 101. Here, the wire 301, which is a first jaw wire, is wound around the pulley 113, and the wire 305, which is a first jaw wire, is wound around the pulley 114.

The pulley 115 and the pulley 116 function as end tool first jaw sub-pulleys. That is, the pulley 115 and the pulley 116 function as sub rotation pulleys for a pitch motion of the first jaw 101. Here, the wire 301, which is a first jaw wire, is wound around the pulley 115, and the wire 305, which is a first jaw wire, is wound around the pulley 116.

Here, the pulley 113 and the pulley 114 are disposed on one side of the pulley 111 and the pulley 112 to face each other. Here, the pulley 113 and the pulley 114 are formed to be rotatable independently of each other around the rotation shaft 143 that is an end tool pitch rotation shaft. In some embodiments, the pulley 115 and the pulley 116 are disposed on one side of the pulley 113 and on one side of the pulley 114, respectively, to face each other. Here, the pulley 115 and the pulley 116 are formed to be rotatable independently of each other around the rotation shaft 144 that is an end tool pitch auxiliary rotation shaft. Here, in the drawings, it is illustrated that the pulley 113, the pulley 115, the pulley 114, and the pulley 116 are all formed to be rotatable around a Y-axis direction, but an embodiment of the present disclosure is not limited thereto, and the rotation axes of the respective pulleys may be formed in various directions according to configurations thereof.

The wire 301, which is a first jaw wire, is sequentially wound to make contact with at least portions of the pulley 115, the pulley 113, and the pulley 111. In some embodiments, the wire 305 connected to the wire 301 by the coupling member 323 is sequentially wound to make contact with at least portions of the pulley 111, the pulley 112, the pulley 114, and the pulley 116 in turn.

Viewed from another perspective, the wires 301 and 305, which are first jaw wires, are sequentially wound to make contact with at least portions of the pulley 115, the pulley 113, the pulley 111, the pulley 112, the pulley 114, and the pulley 116 and are formed to move along the above pulleys while rotating the above pulleys.

Accordingly, when the wire 301 is pulled in the direction of an arrow 301 of FIG. 9, a coupling member (not shown) to which the wire 301 is coupled and the pulley 111 coupled to the coupling member (not shown) are rotated in an arrow L direction of FIG. 9. In contrast, when the wire 305 is pulled in the direction of an arrow 305 of FIG. 9, a coupling member (not shown) to which the wire 305 is coupled and the pulley 111 coupled to the coupling member (not shown) are rotated in an arrow R direction of FIG. 9.

Hereinafter, the pulley 112 and the pulley 122 serving as auxiliary pulleys will be described in more detail.

The pulley 112 and the pulley 122 may serve to increase rotation angles of the first jaw 101 and the second jaw 102, respectively, by coming into contact with the wire 305, which is a first jaw wire, and the wire 302, which is a second jaw wire, and changing the arrangement paths of the wires 305 and 302 to a certain extent.

That is, when the auxiliary pulleys are not disposed, each of the first jaw and the second jaw may be rotated up to a right angle, but in an embodiment of the present disclosure, the pulley 112 and the pulley 122, which are auxiliary pulleys, are additionally provided, so that the maximum rotation angle may be increased by θ as shown in FIG. 9. This enables a motion of the two jaws of the end tool 100 being opened for an actuation motion while the two jaws are yaw-rotated by 90° in the L direction. This is because the second jaw 102 is rotated by the additional angle θ as shown in FIG. 7. Similarly, an actuation motion is possible even when the two jaws are yaw-rotated in the R direction. In other words, a feature of increasing the range of yaw rotation in which an actuation motion is possible may be obtained through the pulley 112 and the pulley 122.

This will be described in more detail as follows.

When the auxiliary pulleys are not disposed, since the first jaw wire is fixedly coupled to the end tool first jaw pulley, and the second jaw wire is fixedly coupled to the end tool second jaw pulley, each of the end tool first jaw pulley and the end tool second jaw pulley may be rotated up to 90°. In this case, when the actuation motion is performed while the first jaw and the second jaw are located at a 90° line, the first jaw may be opened, but the second jaw may not be rotated beyond 90°. Accordingly, when the first jaw and the second jaw perform a yaw motion over a certain angle, there was a problem that the actuation motion is not smoothly performed.

In order to address such a problem, in the multi-joint type surgical instrument 30 according to an embodiment of the present disclosure, the pulley 112 and the pulley 122, which are auxiliary pulleys, are additionally disposed at one side of the pulley 111 and one side of the pulley 121, respectively. As described above, as the arrangement paths of the wire 305, which is a first jaw wire, and the wire 302, which is a second jaw wire, are changed to a certain extent by disposing the pulley 112 and the pulley 122, a tangential direction of the wires 305 and 302 is changed, and accordingly, the coupling member 326 for coupling the wire 302 and the pulley 121 may be rotated up to a line N of FIG. 9. That is, the coupling member 326, which is a coupling part of the wire 302 and the pulley 121, is rotatable until the coupling member 326 is located on a common internal tangent of the pulley 121 and the pulley 122. Similarly, the coupling member 323, which is a coupling part of the wire 305 and the pulley 111, is rotatable until the coupling member 323 is located on a common internal tangent of the pulley 111 and the pulley 112, so that the range of rotation in the L direction may be increased.

In other words, by the pulley 112, the wires 301 and 305, which are two strands of the first jaw wire wound around the pulley 111, are disposed at one side with respect to a plane perpendicular to the Y-axis and passing through the X-axis. Simultaneously, by the pulley 122, the wires 302 and 306, which are two strands of the second jaw wire wound around the pulley 121, are disposed at another side with respect to the plane perpendicular to the Y-axis and passing through the X-axis.

In other words, the pulley 113 and the pulley 114 are disposed at one side with respect to the plane perpendicular to the Y-axis and passing through the X-axis, and the pulley 123 and the pulley 124 are disposed at another side with respect to the plane perpendicular to the Y-axis and passing through the X-axis.

In other words, the wire 305 is located on the internal tangent of the pulley 111 and the pulley 112, and the rotation angle of the pulley 111 is increased by the pulley 112. In some embodiments, the wire 302 is located on the internal tangent of the pulley 121 and the pulley 122, and the rotation angle of the pulley 121 is increased by the pulley 122.

According the above-described embodiment of the present disclosure, as the rotation radii of the jaw 101 and the jaw 102 increase, an effect of increasing a yaw motion range in which a normal opening/closing actuation motion is performed may be obtained.

Next, components related to the rotation of the pulley 121 will be described.

The pulley 123 and the pulley 124 function as end tool second jaw pitch main pulleys. That is, the pulley 123 and the pulley 124 function as main rotation pulleys for a pitch motion of the second jaw 102. Here, the wire 306, which is a second jaw wire, is wound around the pulley 123, and the wire 302, which is a second jaw wire, is wound around the pulley 124.

The pulley 125 and the pulley 126 function as end tool second jaw sub-pulleys. That is, the pulley 125 and the pulley 126 function as sub rotation pulleys for a pitch motion of the second jaw 102. Here, the wire 306, which is a second jaw wire, is wound around the pulley 125, and the wire 302, which is a second jaw wire, is wound around the pulley 126.

On one side of the pulley 121, the pulley 123 and the pulley 124 are disposed to face each other. Here, the pulley 123 and the pulley 124 are formed to be rotatable independently of each other around the rotation shaft 143 that is an end tool pitch rotation shaft. In some embodiments, the pulley 125 and the pulley 126 are disposed on one side of the pulley 123 and one side of the pulley 124, respectively, to face each other. Here, the pulley 125 and the pulley 126 are formed to be rotatable independently of each other around the rotation shaft 144, which is an end tool pitch auxiliary rotation shaft. Here, in the drawings, it is illustrated that all of the pulley 123, the pulley 125, the pulley 124, and the pulley 126 are formed to be rotatable around the Y-axis direction, but an embodiment of the present disclosure is not limited thereto, and the rotation axes of the respective pulleys may be formed in various directions according to configurations thereof.

The wire 306, which is a second jaw wire, is sequentially wound to make contact with at least portions of the pulley 125, the pulley 123, and the pulley 121. In some embodiments, the wire 302 connected to the wire 306 by the coupling member 326 is sequentially wound to make contact with at least portions of the pulley 121, the pulley 122, the pulley 124, and the pulley 126.

Viewed from another perspective, the wires 306 and 302, which are second jaw wires, are sequentially wound to make contact with at least portions of the pulley 125, the pulley 123, the pulley 121, the pulley 122, the pulley 124, and the pulley 126, and are formed to move along the above pulleys while rotating the above pulleys.

Accordingly, when the wire 306 is pulled in the direction of an arrow 306 of FIG. 9, the coupling member 326 to which the wire 306 is coupled and the pulley 121 coupled to the coupling member 326 are rotated in the arrow R direction of FIG. 9. In contrast, when the wire 302 is pulled in the direction of an arrow 302 of FIG. 9, the coupling member 326 to which the wire 302 is coupled and the pulley 121 coupled to the coupling member 326 are rotated in the arrow L direction of FIG. 9.

Hereinafter, a pitch motion of the present disclosure will be described in more detail.

First, for the pitch motion, at the end tool 100 side, the pulley 113, the pulley 114, the pulley 123, and the pulley 124, which are end tool jaw pitch main pulleys, are formed to be rotatable around the rotation shaft 143. Meanwhile, in a direction of the proximal end 105 of the end tool jaw pitch main pulley, the pulley 115, the pulley 116, the pulley 125, and the pulley 126, which are end tool jaw pitch sub-pulleys, are formed to be rotatable around the rotation shaft 144.

In some embodiments, based on a plane perpendicular to the rotation shaft 141 and including the rotation shaft 143 (i.e., an XY plane), the wires 301 and 305, which are two strands of the first jaw wire, are located on the same side with respect to the XY plane That is, the wire 301 and the wire 305 are formed to pass through lower sides of the pulley 113 and the pulley 114, which are end tool jaw pitch main pulleys, and upper sides of the pulley 115 and the pulley 116, which are end tool jaw pitch sub-pulleys.

Similarly, the wires 302 and 306, which are two strands of the second jaw wire, are located on the same side with respect to the XY plane. That is, the wires 302 and 306 are formed to pass through upper sides of the pulley 123 and the pulley 124, which are end tool jaw pitch main pulleys, and lower sides of the pulley 125 and the pulley 126, which are end tool jaw pitch sub-pulleys.

In some embodiments, in the wires 301 and 305 that are two strands of the first jaw wire, when the wire 301 is pulled toward the arrow 301 of FIG. 9 and simultaneously the wire 305 is pulled toward the arrow 305 of FIG. 9 (i.e., when both strands of the first jaw wire are pulled in the same direction), as shown in FIG. 7, since the wires 301 and 305 are wound around lower portions of the pulleys 113 and 114, which are rotatable around the rotation shaft 143 that is an end tool pitch rotation shaft, the pulley 111 to which the wire 301 and the wire 305 are fixedly coupled, and the end tool hub 106 to which the pulley 111 is coupled are rotated together as a whole in a counterclockwise direction around the rotation shaft 143, as a result, the end tool 100 performs the pitch motion while rotating downward. At this time, since the second jaw 102 and the wires 302 and 306 fixedly coupled thereto are wound around the upper portions of the pulleys 123 and 124 rotatable around the rotation shaft 143, the wires 302 and 306 are unwound in opposite directions of the arrows 302 and 306, respectively.

In contrast, in the wires 302 and 306 that are two strands of the second jaw wire, when the wire 302 is pulled toward the arrow 302 of FIG. 9 and simultaneously the wire 306 is pulled toward the arrow 306 of FIG. 9 (i.e., when both strands of the second jaw wire are pulled in the same direction), as shown in FIG. 7, since the wires 302 and 306 are wound around lower portions of the pulleys 123 and 124, which are rotatable around the rotation shaft 143 that is an end tool pitch rotation shaft, the pulley 121 to which the wire 302 and the wire 306 are fixedly coupled, and the end tool hub 106 to which the pulley 121 is coupled are rotated together as a whole in a clockwise direction around the rotation shaft 143, as a result, the end tool 100 performs the pitch motion while rotating upward. At this time, since the first jaw 101 and the wires 301 and 305 fixedly coupled thereto are wound around the lower portions of the pulleys 113 and 114 rotatable around the rotation shaft 143, the wires 302 and 306 are moved in opposite directions of the arrows 301 and 305, respectively.

Viewed from another perspective, it may be also described that both strands of each jaw wire are moved simultaneously in the same direction when the end tool 100 is pitch-rotated.

Meanwhile, the end tool 100 of the multi-joint type surgical instrument 30 of the present disclosure may further include the pulley 131, which is an end tool pitch pulley, the driving part 200 may further include the pulley 231, which is a driving part pitch pulley, and the power transmission part 300 may further include the wire 303 and the wire 304 that are pitch wires. In some embodiments, the pulley 131 of the end tool 100 is rotatable around the rotation shaft 143, which is an end tool pitch rotation shaft, and may be integrally formed with the end tool hub 106 (or fixedly coupled to the end tool hub 106) as one body. In some embodiments, the wires 303 and 304 may serve to connect the pulley 131 of the end tool 100 to the pulley 231 of the driving part 200.

Thus, when the pulley 231 of the driving part 200 is rotated, the rotation of the pulley 231 is transmitted to the pulley 131 of the end tool 100 via the wires 303 and 304, which causes the pulley 131 to also be rotated, and as a result, the end tool 100 performs a pitch motion while rotating.

That is, in the multi-joint type surgical instrument 30 according to an embodiment of the present disclosure, by providing the pulley 131 of the end tool 100, the pulley 231 of the driving part 200, and the wires 303 and 304 of the power transmission part 300 to transmit power for a pitch motion, the driving force for a pitch motion from the driving part 200 may be more completely transmitted to the end tool 100, thereby improving operation reliability.

Here, a diameter of each of the pulley 113, the pulley 114, the pulley 123, and the pulley 124, which are end tool jaw pitch main pulleys, and a diameter of the pulley 131, which is an end tool pitch pulley, may be the same as each other or different from each other. At this time, a ratio of the diameter of the end tool jaw pitch main pulley to the diameter of the end tool pitch pulley may be the same as a ratio of a diameter of a driving part relay pulley of the driving part 200, which will be described later, to a diameter of a driving part pitch pulley. This will be described in detail later.

Hereinafter, the driving part 200 of the multi-joint type surgical instrument 30 of FIG. 6 will be described in more detail.

Referring to FIGS. 10 to 16, the driving part 200 of the multi-joint type surgical instrument 30 according to an embodiment of the present disclosure may include the pulley 211, the pulley 212, a pulley 213, a pulley 214, a pulley 215, a pulley 216, a pulley 217, a pulley 218, a pulley 219, and a pulley 220, which are related to a rotational motion of the first jaw 101 In some embodiments, the driving part 200 may include the pulley 221, the pulley 222, a pulley 223, a pulley 224, a pulley 225, a pulley 226, a pulley 227, a pulley 228, a pulley 229, and a pulley 230, which are related to a rotational motion of the second jaw 102.

Here, the pulleys facing each other are illustrated in the drawings as being formed parallel to each other, but an embodiment of the present disclosure is not limited thereto, and each of the pulleys may be variously formed with a position and a size suitable for the configuration of the driving part.

In some embodiments, the driving part 200 of the multi-joint type surgical instrument 30 according to an embodiment of the present disclosure may further include the pulley 231 serving as a driving part pitch pulley, and a pitch-yaw connector 232 configured to connect the pulley 231 to the above-described jaw pulleys of the driving part.

In some embodiments, the driving part 200 according to an embodiment of the present disclosure may include a rotation shaft 241, a rotation shaft 242, a rotation shaft 243, a rotation shaft 244, a rotation shaft 245, and a rotation shaft 246. Here, the rotation shaft 241 may function as a first jaw rotation shaft of the driving part, and the rotation shaft 242 may function as a second jaw rotation shaft of the driving part. In some embodiments, the rotation shaft 243 may function as a driving part pitch rotation shaft, and the rotation shaft 244 may function as a driving part roll rotation shaft. In some embodiments, the rotation shaft 245 may function as a driving part first jaw auxiliary rotation shaft of the driving part, and the rotation shaft 246 may function as a driving part second jaw auxiliary rotation shaft. Each of the rotation shafts 241, 242, 243, 244, 245, and 246 may be fitted into one or more pulleys, which will be described in detail later.

In some embodiments, the driving part 200 according to an embodiment of the present disclosure may include a motor coupling part 251, a motor coupling part 252, a motor coupling part 253, and a motor coupling part 254. Here, the motor coupling part 251 may function as a first jaw driving motor coupling part, the motor coupling part 252 may function as a second jaw driving motor coupling part, the motor coupling part 253 may function as a pitch driving motor coupling part, and the motor coupling part 254 may function as a roll driving motor coupling part. Here, each of the motor coupling parts 251, 252, 253, and 254 may be provided in the form of a rotatable flat plate, in which one or more coupling holes to which a motor (not shown) may be coupled may be formed.

The motor coupling parts 251, 252, 253, and 254 of the driving part 200 described above are coupled to motors (not shown) formed in the robot arm units 21, 22, and 23, respectively, so that the driving part 200 is operated by driving the motors (not shown).

In some embodiments, the driving part 200 according to an embodiment of the present disclosure may include a gear 261, a gear 262, a gear 263, and a gear 264. Here, the gear 261 and the gear 262 may function as pitch driving gears, and the gear 263 and the gear 264 may function as roll driving gears.

Hereinafter, each component will be described in more detail.

The pulley 211 and the pulley 212 may function as driving part first jaw pulleys, and the pulley 221 and the pulley 222 may function as driving part second jaw pulleys, and these components may be collectively referred to as driving part jaw pulleys.

Here, it is illustrated in the drawings that the pulley 211 is associated with a rotational motion of the first jaw 101 of the end tool 100, and the pulley 221 is associated with a rotational motion of the second jaw 102 of the end tool 100, but an embodiment of the present disclosure is not limited thereto. For example, one group of pulleys in the driving part may be associated with a yaw motion, and one group of pulleys in the driving part may be associated with an actuation motion. Thus, the pulley 211 and the pulley 212 may be collectively referred to as driving part driving pulleys. In some embodiments, in the other pulleys, one group of pulleys may also be associated with a yaw motion, and one group of pulleys may also be associated with an actuation motion.

The pulley 213 and the pulley 214 may function as driving part first jaw auxiliary pulleys, and the pulley 223 and the pulley 224 may function as driving part second jaw auxiliary pulleys, and these components may be collectively referred to as driving part auxiliary pulleys.

The pulley 215 and the pulley 216 may function as driving part first jaw first relay pulleys, and the pulley 217 and the pulley 218 may function as driving part first jaw second relay pulleys, and these components may be collectively referred to as driving part first jaw relay pulleys. Meanwhile, the pulley 225 and the pulley 226 may function as driving part second jaw first relay pulleys, and the pulley 227 and the pulley 228 may function as driving part second jaw second relay pulleys, and these components may be collectively referred to as driving part second jaw relay pulleys. Meanwhile, the pulley 215, the pulley 216, the pulley 225, and the pulley 226 may be collectively referred to as driving part first relay pulleys, and the pulley 217, the pulley 218, the pulley 227, and the pulley 228 may be collectively referred to as driving part second relay pulleys. Furthermore, the pulley 215, the pulley 216, the pulley 217, the pulley 218, the pulley 225, the pulley 226, the pulley 227, and the pulley 228 may be collectively referred to as driving part relay pulleys.

Here, it is illustrated in the drawings that two pulleys are paired to form the driving part relay pulleys for each jaw, but an embodiment of the present disclosure is not limited thereto. For example, it is illustrated that the pulley 215, which is a driving part first jaw first relay pulley, and the pulley 217, which is a driving part first jaw second relay pulley, are formed as a pair, and the wire 301 sequentially passes through the pulley 215 and the pulley 217. However, the driving part first jaw relay pulley may be configured with not just two pulleys but also with three or more pulleys.

Meanwhile, the pulley 219 and the pulley 220 may function as driving part first jaw satellite pulleys, and the pulley 229 and the pulley 230 may function as driving part second jaw satellite pulleys, and these two components may be collectively referred to as driving part satellite pulleys.

A plurality of rotation shafts including the rotation shaft 241, the rotation shaft 242, the rotation shaft 243, the rotation shaft 244, the rotation shaft 245, and the rotation shaft 246 may be formed on a first surface of a base plate 201. In some embodiments, a plurality of relay pulleys 202 are formed on the first surface of the base plate 201, and may serve to redirect the wires 301, 302, 303, 304, 305, and 306 entering the driving part 200 through the connection part 310 toward the pulley 231.

In some embodiments, the connection part 310 in the form of a shaft is coupled to a second surface of the base plate 201 opposite to the first surface, and the motor coupling part 251, the motor coupling part 252, the motor coupling part 253, and the motor coupling part 254, to which the motors (not shown) for driving the pulleys are coupled, may be formed on the second surface.

Here, each rotation shaft and each motor coupling part may be directly connected or indirectly connected to each other via a gear.

As an example, by directly coupling the motor coupling part 251, which is a first jaw driving motor coupling part, to the rotation shaft 241 that is a driving part first jaw rotation shaft, when the motor coupling part 251 coupled to a first jaw driving motor (not shown) is rotated, the rotation shaft 241 directly coupled to the motor coupling part 251 may be rotated together. Similarly, by directly coupling the motor coupling part 252, which is a second jaw driving motor coupling part, to the rotation shaft 242 that is a driving part second jaw rotation shaft, when the motor coupling part 252 coupled to a second jaw driving motor (not shown) is rotated, the rotation shaft 242 directly coupled to the motor coupling part 252 may be rotated together.

In another example, when viewed from a plane perpendicular to the rotation shaft 243, the motor coupling part 253, which is a pitch driving motor coupling part, and the rotation shaft 243, which is a driving part pitch rotation shaft, may be disposed to be spaced apart from each other by a certain extent. In some embodiments, the motor coupling part 253 and the rotation shaft 243 may be connected to each other by the gears 261 and 263, which are pitch driving gears.

Similarly, when viewed from a plane perpendicular to the rotation shaft 244, the motor coupling part 254, which is a roll driving motor coupling part, and the rotation shaft 244, which is a driving part roll rotation shaft, may be disposed to be spaced apart from each other by a certain extent. In some embodiments, the motor coupling part 254 and the rotation shaft 244 may be connected to each other by the gears 263 and 264, which are roll driving gears.

As such, some motor coupling parts are configured to be directly connected to the rotation shafts, respectively, and the remaining motor coupling parts are configured to be indirectly connected to the rotation shafts, respectively, because the coupling position and direction between the multi-joint type surgical instrument 30 and the slave robot 20 should be considered. That is, the rotation shaft that is not affected by the coupling position with the slave robot 20 is directly connected to the motor coupling part, whereas the rotation shaft that may cause interference with the coupling position with the slave robot 20 may be indirectly connected to the motor coupling part.

It is illustrated in the drawings that the motor coupling part 251 and the motor coupling part 252 are directly connected to the rotation shafts, respectively, and the motor coupling part 253 and the motor coupling part 254 are indirectly connected, respectively, through the gears, but an embodiment of the present disclosure is not limited thereto, and various configurations are possible according to the coupling position and direction with the slave robot 20.

The pulleys 211 and 212, which are driving part first jaw pulleys, may be coupled to the rotation shaft 241, which is a driving part first jaw rotation shaft. Here, the pulleys 211 and 212 may be formed to rotate together with the rotation shaft 241.

In some embodiments, the rotation shaft 245, which is a driving part first jaw auxiliary rotation shaft, may be disposed in a region adjacent to the rotation shaft 241. The pulleys 213 and 214, which are driving part first jaw auxiliary pulleys, may be coupled to the rotation shaft 245. Here, the pulleys 213 and 214 may be formed to be rotatable around the rotation shaft 245.

Here, it is illustrated in the drawings that the driving part first jaw pulley is formed of two pulleys 211 and 212, the wire 301 is coupled to one pulley 211, and the wire 305 is coupled to another pulley 212. However, an embodiment of the present disclosure is not limited thereto, and the driving part first jaw pulley may be formed of one pulley, and both the wires 301 and 305 may be coupled to the one pulley.

As described above, the rotation shaft 241 is coupled to the first jaw driving motor (not shown) by the motor coupling part 251, and thus, when the first jaw driving motor (not shown) rotates for driving the first jaw 101, the pulleys 211 and 212, which are driving part first jaw pulleys, are rotated together with the rotation shaft 241, so that the wires 301 and 305, which are first jaw wires, are pulled or released.

The pulleys 221 and 222, which are driving part second jaw rotation shafts, may be coupled to the rotation shaft 242 that is a driving part second jaw pulley. Here, the pulley 221 and the pulley 222 may be formed to rotate together with the rotation shaft 242.

In some embodiments, the rotation shaft 246, which is a driving part second jaw auxiliary rotation shaft, may be disposed in a region adjacent to the rotation shaft 242. The pulleys 223 and 224, which are driving part second jaw auxiliary pulleys, may be coupled to the rotation shaft 245. Here, the pulleys 223 and 224 may be formed to be rotatable around the rotation shaft 246.

Here, it is illustrated in the drawings that the driving part second jaw pulley is formed of two pulleys 221 and 222, the wire 302 is coupled to one pulley 221, and the wire 306 is coupled to another pulley 222. However, an embodiment of the present disclosure is not limited thereto, and the driving part second jaw pulley may be formed of one pulley, and both the wires 302 and 306 may be coupled to the one pulley.

As described above, the rotation shaft 242 is coupled to the second jaw driving motor (not shown) by the motor coupling part 252, and thus, when the second jaw driving motor (not shown) rotates for driving the second jaw 102, the pulley 221 and the pulley 222, which are driving part second jaw pulleys, are rotated together with the rotation shaft 242, so that the wires 302 and 306, which are second jaw wires, are pulled or released.

The pulley 231, which is a driving part pitch pulley, may be coupled to the rotation shaft 243 that is a driving part pitch rotation shaft. Here, the pulley 231 may be formed to rotate together with the rotation shaft 243.

As described above, the rotation shaft 243 is coupled to a pitch driving motor (not shown) by the motor coupling part 253, and thus, when the pitch driving motor (not shown) rotates for a pitch motion, the wires 303 and 304, which are pitch wires, are pulled or released as the pulley 231, which is a driving part pitch pulley, is rotated together with the rotation shaft 243.

Meanwhile, the pulley 215, the pulley 216, the pulley 217, the pulley 218, the pulley 225, the pulley 226, the pulley 227, and the pulley 228, which are driving part relay pulleys, may be formed to be rotatable around the rotation shaft 243 by inserting the rotation shaft 243 therethrough. Here, the pulley 215, the pulley 216, the pulley 217, and the pulley 218, which are driving part first jaw relay pulleys, may be disposed on one surface side of the pulley 231 that is a pitch pulley, and the pulley 225, the pulley 226, the pulley 227, and the pulley 228, which are driving part second jaw relay pulleys, may be disposed on another surface side of the pulley 231.

Viewed from another perspective, along the rotation shaft 243, the pulleys 225 and 226, which are driving part second jaw first relay pulleys, the pulleys 227 and 228, which are driving part second jaw second relay pulleys, the pulley 231, which is a driving part pitch pulley, and the pulleys 217 and 218, which are driving part first jaw second relay pulleys, and the pulleys 215 and 216, which are driving part first jaw first relay pulleys, are sequentially stacked and formed.

In some embodiments, the pitch-yaw connector 232 may be coupled to the rotation shaft 243. The pitch-yaw connector 232 may be formed to rigidly connect the pulley 231, which is a driving part pitch pulley, to the pulley 219, the pulley 220, the pulley 229, and the pulley 230, which are driving part satellite pulleys to allow the driving part satellite pulleys to be revolved around the rotation shaft 243 when the pulley 231 is rotated. This will be described in detail later.

Here, the pitch-yaw connector 232 may be formed to rotate together with the rotation shaft 243. That is, the pulley 231 and the pitch-yaw connector 232 may be coupled to the rotation shaft 243, and may be rotated together with the rotation shaft 243.

Here, the pitch-yaw connector 232 may be described as being formed in an approximately Y-shape as shown in FIG. 12, or the pitch-yaw connector 232 may be described as being formed in a shape in which at least two extension portions 232a and 232b are formed to extend from the center thereof. In some embodiments, a driving part first jaw satellite pulley central shaft 233 and a driving part second jaw satellite pulley central shaft 234 may be formed at end portions of the extension portions 232a and 232b, respectively.

In some embodiments, the pulleys 219 and 220, which are driving part first jaw satellite pulleys, may be coupled to the driving part first jaw satellite pulley central shaft 233, and the pulleys 229 and 230, which are driving part second jaw satellite pulleys, may be coupled to the driving part second jaw satellite pulley central shaft 234.

As a result, when the pulley 231, which is a driving part pitch pulley, is rotated together with the rotation shaft 243, the pulley 219, the pulley 220, the pulley 229, and the pulley 230, which are driving part satellite pulleys, are revolved around the rotation shaft 243. In other words, it may be said that the driving part first jaw satellite pulley central shaft 233 and the driving part second jaw satellite pulley central shaft 234 are rotated around the rotation shaft 243 while maintaining a constant distance from the rotation shaft 243 in a state in which the driving part first jaw satellite pulley central shaft 233 and the driving part second jaw satellite pulley central shaft 234 are spaced apart from the rotation shaft 243 by a certain extent.

That is, the driving part satellite pulley is formed to be movable relative to the driving part relay pulley and the rotation shaft 243 so that a relative position of the driving part satellite pulley with respect to the driving part relay pulley and the rotation shaft 243 may be changed. On the other hand, the relative positions of the driving part pitch pulley and the driving part relay pulley remain constant.

In some embodiments, when the pulley 231, which is a driving part pitch pulley, is rotated around the rotation shaft 243, the pulley 219, the pulley 220, the pulley 229, and the pulley 230, which are driving part satellite pulleys, are moved relative to the pulley 231, which is a driving part pitch pulley, so that the overall lengths of the wire 301, the wire 302, the wire 305, and the wire 306, which are jaw wires, in the driving part 200 are changed.

The wire 301, which is a first jaw wire, is connected to the end tool 100 through the connection part 310 after being sequentially wound to make contact with at least portions of the pulley 211, the pulley 213, the pulley 215, the pulley 219, and the pulley 217 in a state in which one end portion of the wire 301 is coupled to the pulley 211 by the first jaw wire-driving part coupling member (not shown).

Viewed from another perspective, the wire 301, which is a first jaw wire, is connected to the end tool 100 through the connection part 310 after being sequentially passing through the driving part first jaw pulley 211, the driving part first jaw auxiliary pulley 213, the driving part first jaw first relay pulley 215, the driving part first jaw satellite pulley 219, and the driving part first jaw second relay pulley 217.

Viewed from another perspective, the wire 301, which is a first jaw wire, enters the driving part 200 after passing through the end tool 100 and the connection part 310, and then is fixedly coupled to the pulley 211, which is a driving part first jaw pulley after being sequentially wound around the pulley 217, the pulley 219, the pulley 215, and the pulley 213.

Meanwhile, the wire 305, which is a first jaw wire, is connected to the end tool 100 through the connection part 310 after being sequentially wound to make contact with at least portions of the pulley 212, the pulley 214, the pulley 216, the pulley 220, and the pulley 218 in a state in which one end portion of the wire 305 is coupled to the pulley 212 by the first jaw wire-driving part coupling member (not shown).

The wire 302, which is a second jaw wire, is connected to the end tool 100 through the connection part 310 after being sequentially wound to make contact with at least portions of the pulley 221, the pulley 223, the pulley 225, the pulley 229, and the pulley 227 in a state in which one end portion thereof is coupled to the pulley 221 by the second jaw wire-driving part coupling member (not shown).

Meanwhile, the wire 306, which is a second jaw wire, is connected to the end tool 100 through the connection part 310 after being sequentially wound to make contact with at least portions of the pulley 222, the pulley 224, the pulley 226, the pulley 230, and the pulley 228 in a state in which one end portion thereof is coupled to the pulley 222 by the second jaw wire-driving part coupling member (not shown).

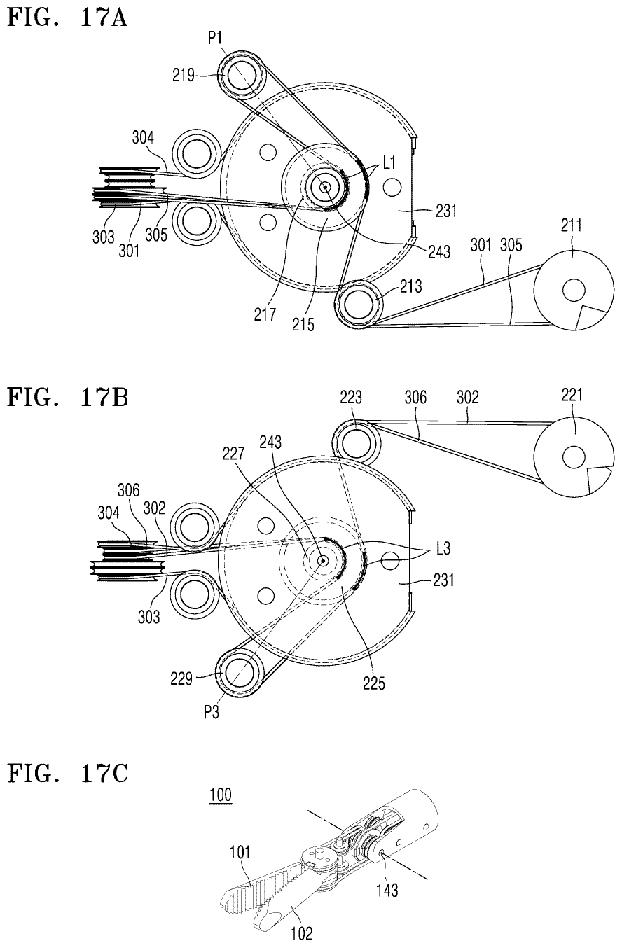

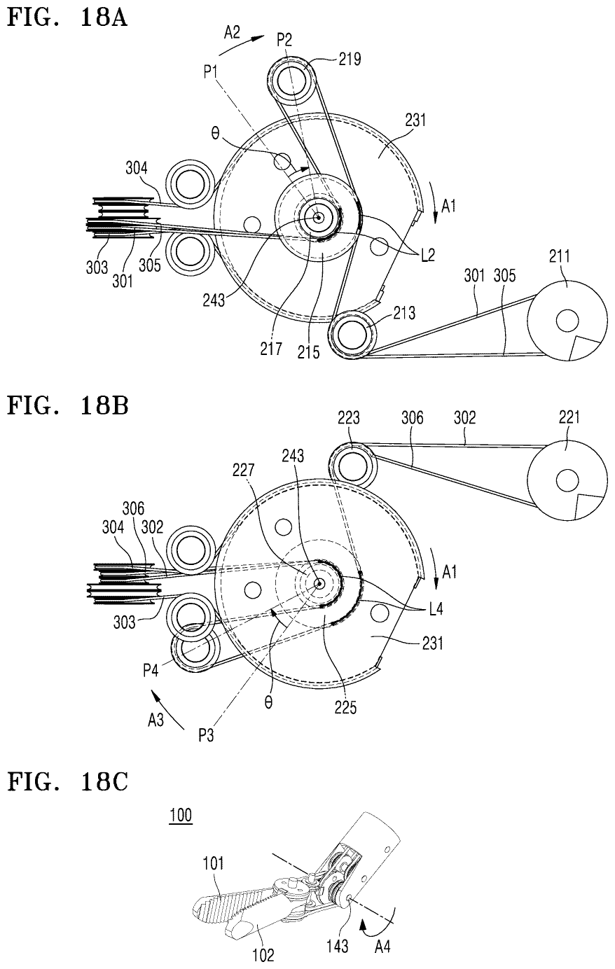

FIGS. 17 and 18 are views illustrating a pitch motion of the multi-joint type surgical instrument illustrated in FIG. 6. Here, for convenience of description, only the pulleys and wires related to the rotation of the first jaw are illustrated in FIGS. 17A and 18A, and only the pulleys and wires related to the rotation of the second jaw are illustrated in FIGS. 17B and 18B. In some embodiments, FIGS. 17C and 18C illustrate a pitch motion of the end tool according to a pitch motion of the driving part.

Here, in the multi-joint type surgical instrument 30 according to an embodiment of the present disclosure, when the driving part satellite pulley is moved relative to the driving part relay pulley, which causes the overall length of the jaw wire to be changed in the driving part 200, allowing the end tool 100 to perform a pitch motion. In particular, in the multi-joint type surgical instrument 30 according to an embodiment of the present disclosure, when the driving part pitch pulley is rotated, which causes the driving part satellite pulley to be revolved around the (common) rotation shaft of the driving part relay pulley and the driving part pitch pulley so that a path length of the jaw wire wound around the driving part relay pulley is changed, allowing the end tool to perform a pitch motion.

In some embodiments, when a motion compensation for the pitch motion is not separately performed in the driving part, the pitch motion itself cannot be performed in the end tool.

Meanwhile, in order for the end tool to perform a pitch motion, the wires 301 and 305 should be further wound around the pulley 113 by ΔS and the wires 302 and 306 should be further unwound from the pulley 114 by ΔS. However, when such compensation is not performed in the driving part, the pitch motion itself cannot be performed in the end tool.

In order to perform motion compensation for the pitch motion as described above, in the multi-joint type surgical instrument 30 according to an embodiment of the present disclosure, the driving part pitch pulleys are rotated while the driving part satellite pulleys are revolved, so that the jaw wires are wound around or released from the driving part relay pulley, which allows the movement of the jaw wires to be compensated for by the rotation of the driving part pitch pulley.

In other words, when the pulley 231, which is a driving part pitch pulley, is rotated together with the rotation shaft 243, the driving part satellite pulleys are revolved around the rotation shaft 243. In some embodiments, as the driving part satellite pulleys are revolved around the rotation shaft 243, the jaw wire wound around the driving part relay pulley is changed in length. That is, the jaw wire wound at the end tool 100 side due to the rotation of the pulley 231 is released by the same amount at the driving part 200 side, and the jaw wire unwound at the end tool 100 side is wound by the same amount at the driving part 200 side, so that the pitch motion does not affect the yaw motion.

Viewed from another perspective, when the end tool performs a pitch motion due to the rotation of the driving part pitch pulley, the jaw wire (responsible for the yaw and actuation motions) is also moved by the pitch motion. That is, as the pitch rotation is performed around the rotation shaft 143 of the end tool 100, both strands of the jaw wire coupled to one jaw are pulled, and both strands thereof coupled to another jaw are released. Accordingly, it may be described that in the present disclosure, in order to compensate for the movement of the jaw wire, when the end tool performs the pitch motion, the overall length of the jaw wire in the driving part is changed while the driving part satellite pulley is moved relative to the driving part relay pulley, so that the jaw wire is released (or pulled) at the end tool side as much as the jaw wire is pulled (or released) at the driving part side, thereby compensating for the movement of the jaw wire when the end tool performs the pitch motion.

Hereinafter, the pitch motion will be described in more detail.

When the pulley 231, which is a driving part pitch pulley, is rotated in the direction of an arrow A1 (i.e., in the clockwise direction in the drawing) in order for the pitch motion, the pitch-yaw connector 232 (see FIG. 10) is rotated in the direction of the arrow A1 together with the pulley 231, and thus, the pulleys 219 and 220, which are driving part satellite pulleys fixedly coupled to the pitch-yaw connector 232 (see FIG. 10), are revolved as a whole in the direction of an arrow A2 of FIG. 18A (i.e., in the clockwise direction in the drawing) around the rotation shaft 243 by θ. That is, when the pulley 231 is rotated, the pulleys 219 and 220 are revolved by θ from the position of P1 of FIG. 17A to the position of P2 of FIG. 18A. Viewed from another perspective, it may be described that when the driving part pitch pulley is rotated, the driving part satellite pulley is moved in conjunction with the driving part pitch pulley.

At the same time, when the pulley 231, which is a driving part pitch pulley, is rotated in the direction of the arrow A1 (i.e., in the clockwise direction in the drawing), the pitch-yaw connector 232 (see FIG. 10) is rotated in the direction of the arrow A1 together with the pulley 231, and thus, the pulleys 229 and 230, which are driving part satellite pulleys fixedly coupled to the pitch-yaw connector 232 (see FIG. 10), are revolved as a whole in the direction of an arrow A3 of FIG. 18B (i.e., in the clockwise direction in the drawing) around the rotation shaft 243 by θ. That is, when the pulley 231 is rotated, the pulleys 229 and 230 are revolved by θ from the position of P3 of FIG. 17B to the position of P4 of FIG. 18B. Viewed from another perspective, it may be described that when the driving part pitch pulley is rotated, the driving part satellite pulley is moved in conjunction with the driving part pitch pulley.

Meanwhile, in this case, the positions of the pulley 215, the pulley 216, the pulley 217, the pulley 218, the pulley 225, the pulley 226, the pulley 227, and the pulley 228, which are driving part relay pulleys coupled to the rotation shaft 243, are not changed. That is, the relative positions of the pulley 211, which is a driving part jaw pulley, the pulley 231, which is a driving part pitch pulley, and the pulley 215, the pulley 216, the pulley 217, and the pulley 218, which are driving part relay pulleys, remain constant. Similarly, the relative positions of the pulley 221, which is a driving part jaw pulley, the pulley 231, which is a driving part pitch pulley, and the pulley 225, the pulley 226, the pulley 227, and the pulley 228, which are driving part relay pulleys, remain constant.

In some embodiments, as described above, the relative position of the driving part satellite pulley with respect to the driving part relay pulley is changed as the driving part satellite pulley is revolved, and thus, the length of each wire wound around the driving part relay pulley, that is, the path length, is changed. Here, since the driving part relay pulley includes the pulley 215, which is a driving part first jaw first relay pulley, and the pulley 217, which is a driving part first jaw second relay pulley, the path length also means the sum of the length of the wire 301 wound around the pulley 215 and the length of the wire 301 wound around the pulley 217 (or, the sum of the length by which the wire 305 is wound around the pulley 216 and the length by which the wire 305 is wound on the pulley 218).

That is, as compared to a path length L1 by which the wires 301 and 305, which are first jaw wires, wound around the driving part relay pulleys at the position of FIG. 17A, a path length L2 by which the first jaw wires wound around the driving part relay pulleys at the position of FIG. 18A is reduced, and thus, the first jaw wires are further released at the driving part 200 side by the reduced path length (L1-L2). That is, the overall lengths of the wires 301 and 305, which are first jaw wires, in the driving part 200 are reduced. In some embodiments, as the overall length of the first jaw wire in the driving part 200 is reduced, the overall length of the first jaw wire in the end tool 100 is increased as much as the first jaw wire is unwound.

In contrast, when the pulley 231, which is a driving part pitch pulley, is rotated in the direction of the arrow A1, as compared to a path length L3 by which the wires 302 and 306, which are second jaw wires, wound around the driving part relay pulleys at the position of FIG. 17B, a path length L4 by which the second jaw wires wound around the driving part relay pulleys at the position of FIG. 18B is increased, and the second jaw wires are further pulled at the driving part 200 side by as much as the increased path length (L4-L3). That is, the overall lengths of the wires 302 and 306, which are second jaw wires, in the driving part 200 are increased. In some embodiments, as the overall length of the second jaw wire in the driving part 200 is increased, the overall length of the second jaw wire in the end tool 100 is reduced as much as the second jaw wire is pulled.

As such, when the pulley 231, which is a driving part pitch pulley, is rotated in the direction of the arrow A1 for a pitch motion, the relative position of the driving part satellite pulley is changed as the driving part satellite pulley is moved relative to the driving part pitch pulley and the driving part relay pulley. In some embodiments, due to the relative movement of the driving part satellite pulley, the overall length of the first jaw wire in the driving part 200 is reduced, and the overall length of the first jaw wire in the end tool 100 is increased. At the same time, due to the relative movement of the driving part satellite pulley, the overall length of the second jaw wire in the driving part 200 is increased, and the overall length of the second jaw wire in the end tool 100 is reduced.

As a result, when the pulley 231, which is a driving part pitch pulley, is rotated in the direction of the arrow A1, the wires 301 and 305, which are two strands of the first jaw wire, are released and the wires 302 and 306, which are two strands of the second jaw wire, are pulled when viewed from the end tool 100 side, so that the end tool 100 performs a pitch motion in the direction of an arrow A4 around the rotation shaft 143.

Here, the term “path length” may be defined as a length of the jaw wire from a point at which the jaw wire enters the driving part first relay pulley to a point at which the jaw wire exits from the driving part second relay pulley through the driving part satellite pulley. That is, the path length may be defined as a length of the wire 301, which is a jaw wire, from a point at which the jaw wire enters the pulley 215, which is a driving part first relay pulley, to a point at which the jaw wire exits from the pulley 217, which is a driving part second relay pulley, through the pulley 219 that is a driving part satellite pulley.

Viewed from another perspective, the path length may be defined as the length of the jaw wire from an initial contact point of the jaw wire with the driving part relay pulley to a final contact point of the jaw wire with the driving part relay pulley on a deployment path of the jaw wire that connects the end tool jaw pulley to the driving part jaw pulley. That is, the path length may be defined as the length of the jaw wire from an initial contact point of the wire 301, which is a jaw wire, with the pulley 215, which is a driving part first relay pulley, to a final contact point of the wire 301 with the pulley 217, which is a driving part second relay pulley.

Meanwhile, as the above-described path length is changed while the driving part satellite pulley is moved relative to the driving part relay pulley, the overall length of the jaw wire in the driving part 200 is also changed. In some embodiments, as the overall length of the jaw wire in the driving part 200 is changed, the overall length of the jaw wire in the end tool 100 is also changed. However, it may be said that since the overall length of the jaw wire in the end tool 100 is also increased (or reduced) by as much as the overall length of the jaw wire increased (reduced) in the driving part 200, a total length of the jaw wire is not changed (assuming that clastic deformation or the like is not considered).

As a result, when the driving part pitch pulley is rotated, the wire 301/wire 305, which are first jaw wires, are released at the driving part 200 side by as much as the wire 301/wire 305, which are first jaw wires, are pulled at the end tool 100 side, as a result, a pitch motion is enabled.

Meanwhile, as described above, the end tool 100 of the multi-joint type surgical instrument 30 of the present disclosure may further include the pulley 131, which is an end tool pitch pulley, the driving part 200 may further include the pulley 231, which is a driving part pitch pulley, and the power transmission part 300 may further include the wire 303 and the wire 304 which are pitch wires.

Accordingly, when the pulley 231, which is a driving part pitch pulley, is rotated in the direction of the arrow A1, due to the rotation of the pulley 231, the wire 304 is wound around the pulley 231 and the wire 303 is released from the pulley 231. Accordingly, the pulley 131, which is an end tool pitch pulley connected to another side of each of the wires 303 and 304, is rotated in the direction of the arrow A2 around the rotation shaft 143, so that the pitch motion may be more surely and reliably performed.

Here, among the pulleys that are rotated around the rotation shaft 143, which is an end tool pitch rotation shaft, the pulley 131, which is an end tool pitch pulley in contact with the wires 303 and 304 that are pitch wires, may be formed to have a diameter different from those of the pulley 113, the pulley 114, the pulley 123, and the pulley 124, which are end tool jaw pitch main pulleys in contact with the wire 301, the wire 305, the wire 302, and the wire 306 that are jaw wires.

In this case, when the rotation shaft 143 is rotated, the lengths of the wires wound around or unwound from the respective pulleys are different from each other. For example, when a diameter of the end tool pitch pulley is 6φ, a diameter of the end tool jaw pitch main pulley is 4φ, and the rotation shaft 143 is rotated by 90°, a length of the pitch wire wound around the end tool pitch pulley is 1.5π, whereas a length of the jaw wire wound around the end tool jaw pitch main pulley may be 1π.

From this perspective, the length of the wire wound around or unwound from the pulley may be defined as “rotation amount.” The rotation amount is a concept different from a rotation angle, and may be calculated as (diameter*rotation angle/360°*π).

In this case, since essentially the pulley 231, which is a driving part pitch pulley, is directly connected to the pulley 131, which is an end tool pitch pulley, by the wires 303 and 304, which are pitch wires, the rotation amount of the driving part pitch pulley is the same as that of the end tool pitch pulley. That is, the pitch wire is released from or wound around the end tool pitch pulley by as much as the pitch wire is wound around or released from the driving part pitch pulley.

Meanwhile, a relation of (diameter of end tool pitch pulley:diameter of end tool jaw pitch main pulley)=(rotation amount of wire wound around end tool pitch pulley: rotation amount of wire wound around end tool jaw pitch main pulley) may be established.

As described above, when, in the end tool 100, the length of the pitch wire wound around the end tool pitch pulley is different from the length of the jaw wire wound around the end tool jaw pitch main pulley, in the driving part 200, the length of the pitch wire to be released should be different from the length of the jaw wire to be released by the same proportion.

To this end, the relationship of (diameter of end tool pitch pulley:diameter of end tool jaw pitch main pulley)=(diameter of driving part pitch pulley:diameter of driving part relay pulley) may be established.

For example, when a ratio of (diameter of end tool pitch pulley:diameter of end tool jaw pitch main pulley) is 6:4, a ratio of (diameter of driving part pitch pulley:diameter of driving part relay pulley) may also be 6:4. According to this ratio, the diameter of the driving part pitch pulley may be 9φ, and the diameter of the driving part relay pulley may be 6φ.

However, here, the driving part relay pulley may include two or more pulleys including the driving part first relay pulley and the driving part second relay pulley. In some embodiments, the sum of the diameters of the driving part first relay pulley and the driving part second relay pulley may be defined as the diameter of the driving part relay pulley.

For example, when the diameter of the driving part relay pulley is 60, there are several possible combinations for (diameter of driving part first relay pulley, diameter of driving part second relay pulley), including (1φ, 5φ), (2φ, 4φ), (3φ, 3φ), (4φ, 2φ), and (5φ, 1φ), among others.” Here, it is illustrated in the drawings that the diameter of the pulley 215, which is a driving part first relay pulley, is 4φ, and the diameter of the pulley 217, which is the driving part second relay pulley, is 2φ.

In some embodiments, it may be described that (rotation amount of driving part first relay pulley+rotation amount of driving part second relay pulley) is proportional to the rotation amount of the driving part pitch pulley.

However, although the ratio of (diameter of end tool pitch pulley:diameter of end tool jaw pitch main pulley) does not exactly match the ratio of (diameter of driving part pitch pulley:diameter of driving part relay pulley), when the pulley diameters are selected to make these ratios similar, the object of the present disclosure, which is to compensate for the movement of the jaw wire with the rotation of the driving part pitch pulley, can be achieved to some extent. The process of the final pitch motion will be described again as follows.

Hereinafter, a case in which the diameter of the end tool pitch pulley is 6φ, the diameter of the end tool jaw pitch main pulley is 4φ, the diameter of the driving part pitch pulley is 9φ, and the diameter of the driving part relay pulley is 6φ will be described as an example.

First, for a pitch motion, the pulley 231, which is a driving part pitch pulley of the driving part 200, is rotated by 60° to wind the wire 304, which is a pitch wire, while releasing the wire 303. At this time, the length of the wire 303/wire 304 wound and unwound is 1.5π.

Accordingly, as the wire 304 is pulled by 1.5π and the wire 303 is released by 1.5π in the end tool 100, the pulley 131, which is an end tool pitch pulley, is rotated by 90° corresponding to 1.5π.

Meanwhile, when the pulley 131 is pitch-rotated around the rotation shaft 143, the jaws 101 and 102 and the pulley 111/pulley 112 are also pitch-rotated around the rotation shaft 143. Accordingly, the wires 301 and 305, which are first jaw wires coupled to the pulley 111, are both pulled, and the wires 302 and 306, which are second jaw wires coupled to the pulley 121, are both released. At this time, the angles by which the end tool pitch pulley and the end tool jaw pitch main pulley are rotated are equal to each other and measure 90°, and thus, the length of the jaw wires wound around or released from the end tool jaw pitch main pulley becomes 1π.

Meanwhile, since the pulley 231 and the pulley 219/pulley 220 are rigidly connected by the pitch-yaw connector 232, when the pulley 231 is rotated by 60° around the rotation shaft 243, the pulley 219/pulley 220 are revolved by 60° around the rotation shaft 243.

In some embodiments, as described above, as the pulley 219/pulley 220 are revolved, the jaw wires are wound around or released from the pulley 215 and the pulley 216, whose combined diameter is 6φ, by 1π corresponding to a revolution angle of 60°. That is, the wires 301 and 305, which are first jaw wires, are released as a whole, and the wires 302 and 306, which are second jaw wires, are pulled as a whole.

In other words, the overall path lengths of the wires 301 and 305 wound around the pulley 215, the pulley 216, the pulley 217, and the pulley 218, which are driving part first jaw relay pulleys, are reduced, and the wires 301 and 305 are released by as much as the reduced path length. In some embodiments, the overall path lengths of the wires 302 and 306 wound around the pulley 225, the pulley 226, the pulley 227, and the pulley 228, which are driving part second jaw relay pulleys, are increased, and the wires 302 and 306 are pulled by as much as the increased path length.

That is, the wires 301 and 305, which are first jaw wires, are released at the driving part 200 side by as much as the wires 301 and 305 are pulled at the end tool 100 side, thereby compensating for the movement of the jaw wire due to the pitch motion. Similarly, the wires 302 and 306, which are second jaw wires, are released at the driving part 200 side by as much as the wires 302 and 306 are pulled at the end tool 100 side, thereby compensating for the movement of the jaw wire due to the pitch motion.

As a result, by releasing (or pulling) the jaw wires at the driving part 200 side by as much as a length equal to the length by which the jaw wires are wound around (or released from) the end tool 100 side in response to the pitch motion, the pitch motion can be performed independently without affecting the rotation of the jaw around the yaw shaft.

That is, when the driving part pitch pulley and the driving part satellite pulley are rigidly connected, and the driving part pitch pulley is rotated around the rotation shaft 243, the path length of the jaw wire wound around the driving part relay pulley is changed as the driving part satellite pulley is revolved around the rotation shaft 243. In some embodiments, the change in the path length of the jaw wire compensates for the movement of the jaw wires at the end tool side due to the pitch motion, as a result, the pitch motion is independently performed.

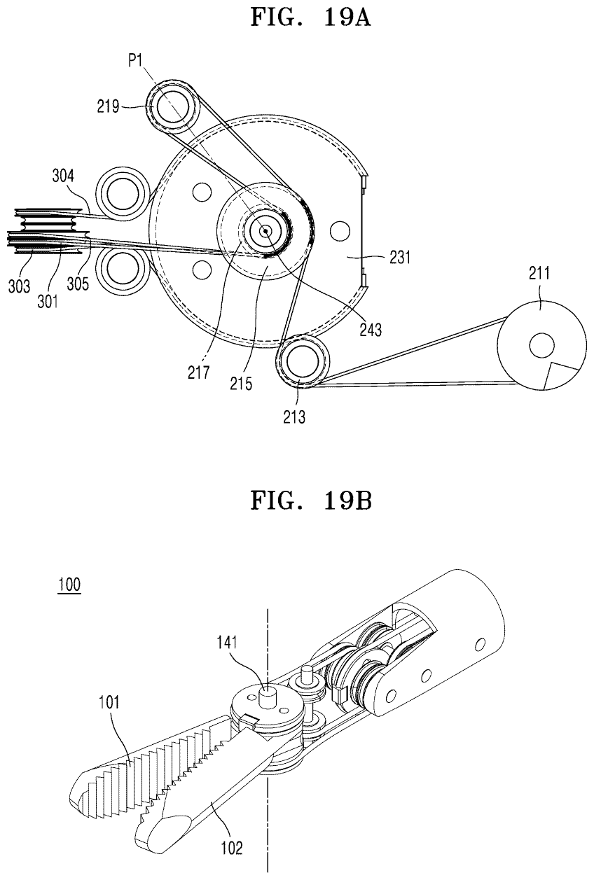

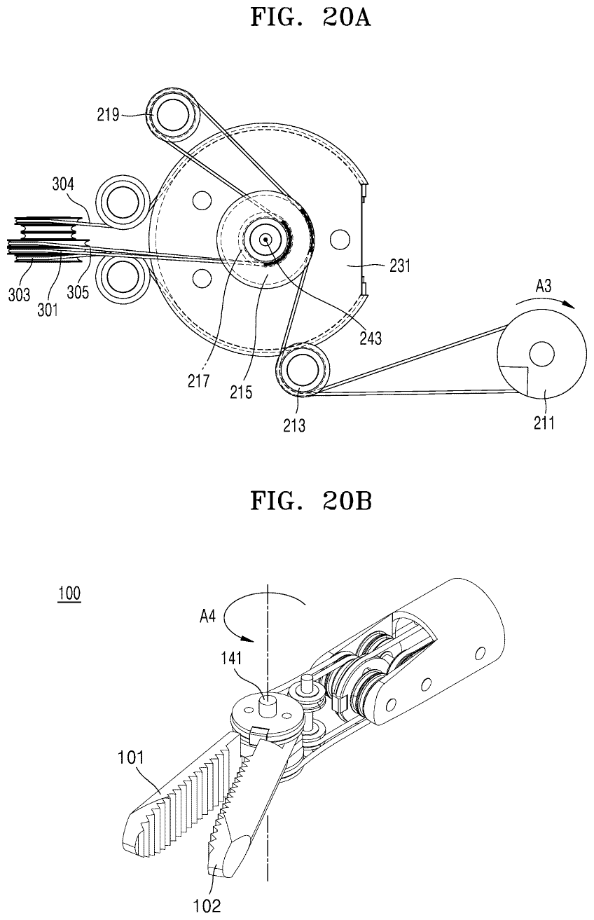

FIGS. 19 and 20 are views illustrating a yaw motion of the multi-joint type surgical instrument illustrated in FIG. 6.

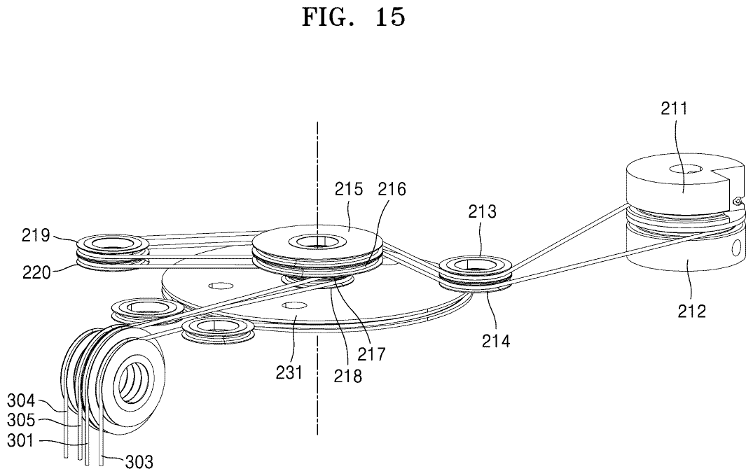

Referring to FIGS. 15, 16, 19, and 20 and the like, when the pulley 211, which is a driving part first jaw pulley, is rotated in the direction of an arrow A3 for a yaw motion, one of the wires 301 and 305, which are first jaw wires, is wound around the pulley 211 and another one thereof is released from the pulley 211 in response to the rotation of the pulley 211. Accordingly, the pulley 111, which is an end tool first jaw pulley connected to the opposite side of the wires 301 and 305, is rotated in the direction of as arrow A4, so that the yaw motion is performed.

At this time, the pulley 219, the pulley 220, the pulley 229, and the pulley 230, which are driving part satellite pulleys, and the pulley 215, the pulley 216, the pulley 217, the pulley 218, the pulley 225, the pulley 226, the pulley 227, and the pulley 228, which are driving part relay pulleys, are not changed in position, but only the motion in which the wires 301 and 305 are wound around or released from the driving part satellite pulley and the driving part relay pulley occurs.

Accordingly, the driving part pitch pulley rigidly connected to the driving part satellite pulley is not rotated, and the wires 303 and 304, which are pitch wires, are not wound or released and maintained in position.

Similarly, when the pulley 221, which is a driving part second jaw pulley, is rotated for a yaw motion, in response to the rotation of the pulley 221, one of the wires 302 and 306, which are second jaw wires, is wound around the pulley 221 and another one thereof is released from the pulley 221. Accordingly, the pulley 121, which is an end tool second jaw pulley connected to the opposite side of the wires 302 and 306, is rotated in one direction, so that the yaw motion is performed.

At this time, the pulley 219, the pulley 220, the pulley 229, and the pulley 230, which are driving part satellite pulleys, and the pulley 215, the pulley 216, the pulley 217, the pulley 218, the pulley 225, the pulley 226, the pulley 227, and the pulley 228, which are driving part relay pulleys, are not changed in position, but only the motion in which the wires 302 and 306 are wound around or released from the driving part satellite pulley and the driving part relay pulley occurs.

Accordingly, the driving part pitch pulley rigidly connected to the driving part satellite pulley is not rotated, and the wires 303 and 304, which are pitch wires, are not wound or released and maintained in position.

As a result, the overall lengths of the wire 301, the wire 302, the wire 305, and the wire 306, which are jaw wires, in the driving part 200 remain constant even when the pulley 211 or pulley 221, which is a driving part jaw pulley, is rotated for the yaw or actuation motion.

As described above, in the multi-joint type surgical instrument 30 according to an embodiment of the present disclosure, when the driving part pitch pulley is rotated, the driving part satellite pulley is revolved around the rotation shaft of the driving part pitch pulley to change the path length of the jaw wire wound around the driving part relay pulley, and the jaw wire is wound or released in response to the rotation of the driving part pitch pulley, so that the movement of the jaw wire due to the pitch drive may be offset or compensated, and as a result, the effect of separating the pitch motion and the yaw motion can be obtained.

However, the pitch motion and the yaw motion are not limited to being mechanically separated from each other as described above, and can be separated and performed independently by the processor according to an embodiment of the present disclosure.

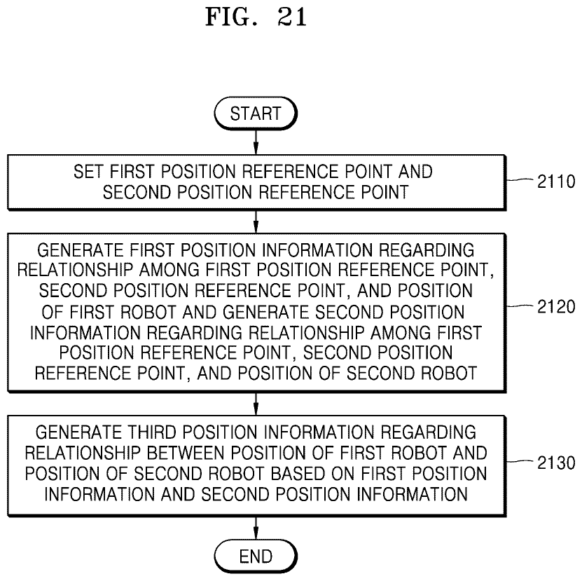

FIG. 21 is a flowchart for describing an example of a method of driving a surgical instrument according to an embodiment.

Referring to FIG. 21, at operation 2110, the processor 2011 sets a first position reference point and a second position reference point.

At each of the first position reference point and the second position reference point, the components of the first robot and the components of the second robot may be located or may be located through movement. For example, the first-1 component of the first robot and the second-1 component of the second robot may be located at the first position reference point, and the first-2 component of the first robot and the second-2 component of the second robot may be located at the second position reference point.

The first-1 component, the first-2 component, the second-1 component, and the second-2 component may be some components of the robot. For example, the first-1 component, first-2 component, second-1 component, and second-2 component may be some components of the robot arm, some components of an end effector, or devices (e.g., rods) additionally mounted on the robot, but the present disclosure is not limited thereto.

At operation 2120, the processor 2011 may generate first position information regarding a relationship among the first position reference point, the second position reference point, and a position of the first robot. In some embodiments, the processor 2011 may generate second position information regarding a relationship among the first position reference point, the second position reference point, and a position of the second robot.

The first position information may include position information generated based on the first position reference point, the second position reference point, and the position of the first robot. As an example, the first position information may include relative position information of the first robot, which is determined based on the first position reference point and the second position reference point. As another example, the first position information may include vector values with respect to the first and second position reference points, using the position of the first robot as the origin. However, examples of the first position information are not limited to those described above.

The second position information may include position information generated based on the first position reference point, the second position reference point, and the position of the second robot. As an example, the second position information may include relative position information of the second robot, which is determined based on the first position reference point and the second position reference point. As another example, the second position information may include vector values for to the first and second position reference points, with the position of the second robot as the origin. However, examples of second position information are not limited to those described above.

As an example, the processor 2011 may calculate a first vector value for the first position reference point and the second position reference point based on the position of the first robot. In some embodiments, the processor 2011 may calculate a second vector value for the first position reference point and the second position reference point based on the position of the second robot. Here, the first position information may include the first vector value, and the second position information may include the second vector value.

As another example, the processor 2011 may calculate a first-1 intermediate vector value based on the first position reference point and the position of the first robot. In some embodiments, the processor 2011 may calculate a first-2 intermediate vector value based on the second position reference point and the position of the first robot. In some embodiments, the processor 2011 may calculate the first vector value based on the first-1 intermediate vector value and the first-2 intermediate vector value. In some embodiments, the processor 2011 may calculate a second-1 intermediate vector value based on the first position reference point and the position of the second robot. In some embodiments, the processor 2011 may calculate a second-2 intermediate vector value based on the second position reference point and the position of the second robot. In some embodiments, the processor 2011 may calculate the second vector value based on the second-1 intermediate vector value and the second-2 intermediate vector value.

At operation 2130, the processor 2011 may generate third position information regarding a relationship between the position of the first robot and the position of the second robot based on the first position information and the second position information.

For example, the processor 2011 may generate the third position information regarding the relationship between the position of the first robot and the position of the second robot based on a correlation between the first position information and the second position information. Here, the third position information may include a relative angle generated based on the position of the first robot and the position of the second robot.

Meanwhile, the processor 2011 may control at least one of the first robot and the second robot based on the third position information. For example, the processor 2011 may control motions of the surgical instrument, which are included in a video image captured by a camera mounted on the first robot, based on the third position information. The surgical instrument may be mounted on the second robot. At this time, the motion of the master robot manipulated by the user aligns with the motion, position, and orientation of the surgical instrument as shown in the video image captured by the camera, thereby enabling the user to intuitively control the surgical instrument.

As an example, the processor 2011 may generate driving information based on the operations of the master robot controlling the first robot and the second robot, and on a transformation relationship among the position of the first robot equipped with a camera, the position of the second robot equipped with a surgical instrument, and the position of the surgical instrument. At this time, the processor 2011 may generate the driving information using the third position information. In some embodiments, the processor 2011 may control at least one of the first robot and the second robot based on the driving information.

As another example, the processor 2011 may generate first intermediate driving information based on a transformation relationship between a position of an image captured by the camera and the position of the first robot, and operation information of the master robot. In some embodiments, the processor 2011 may generate second intermediate driving information based on a transformation relationship between the position of the first robot and the position of the second robot and the first intermediate driving information. In some embodiments, the processor 2011 may generate the driving information based on the transformation relationship between the position of the surgical instrument and the position of the second robot and the second intermediate driving information.

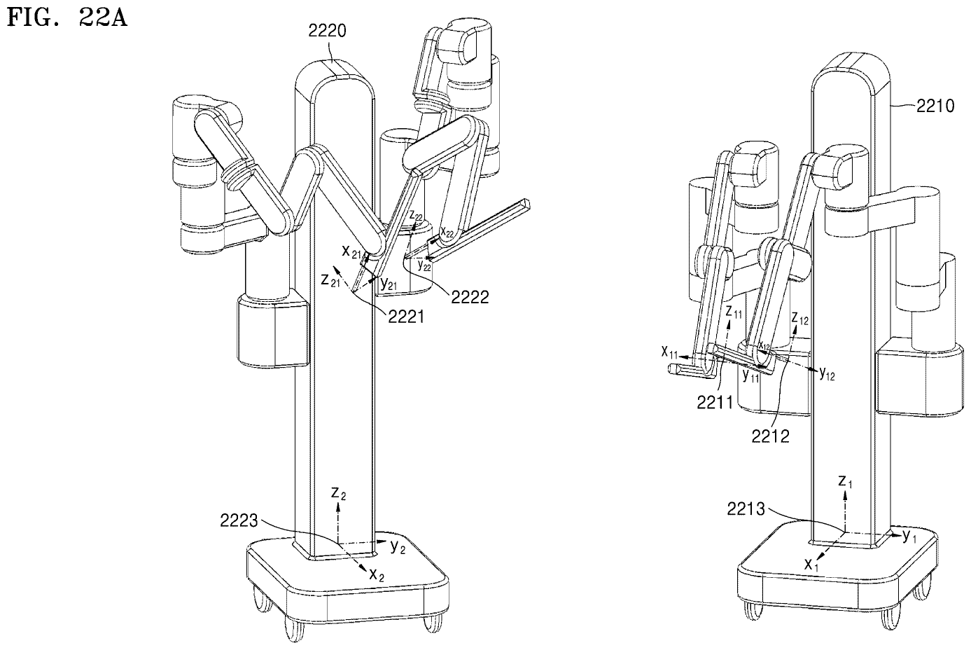

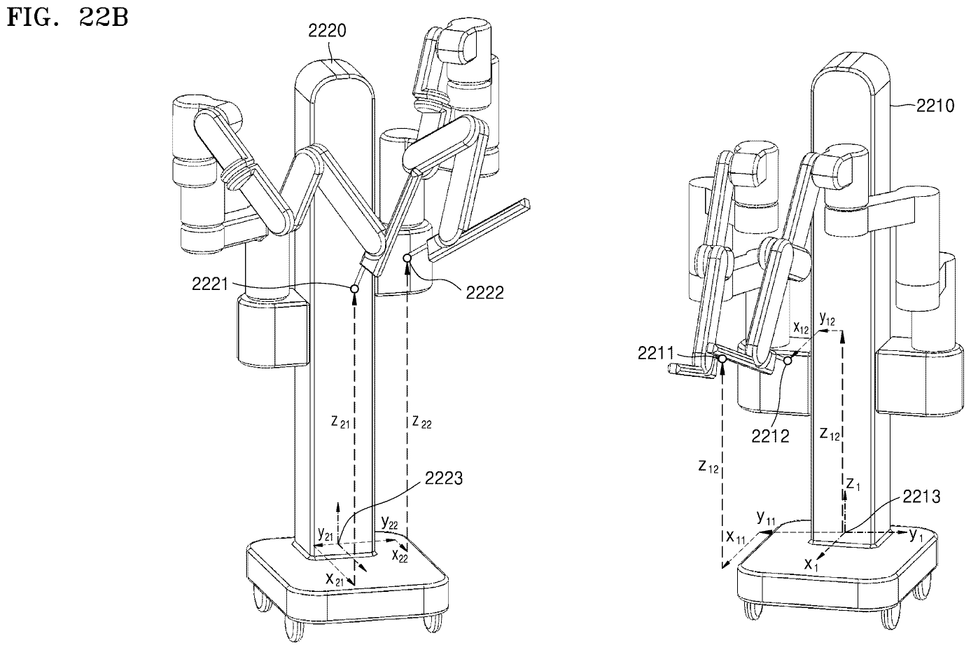

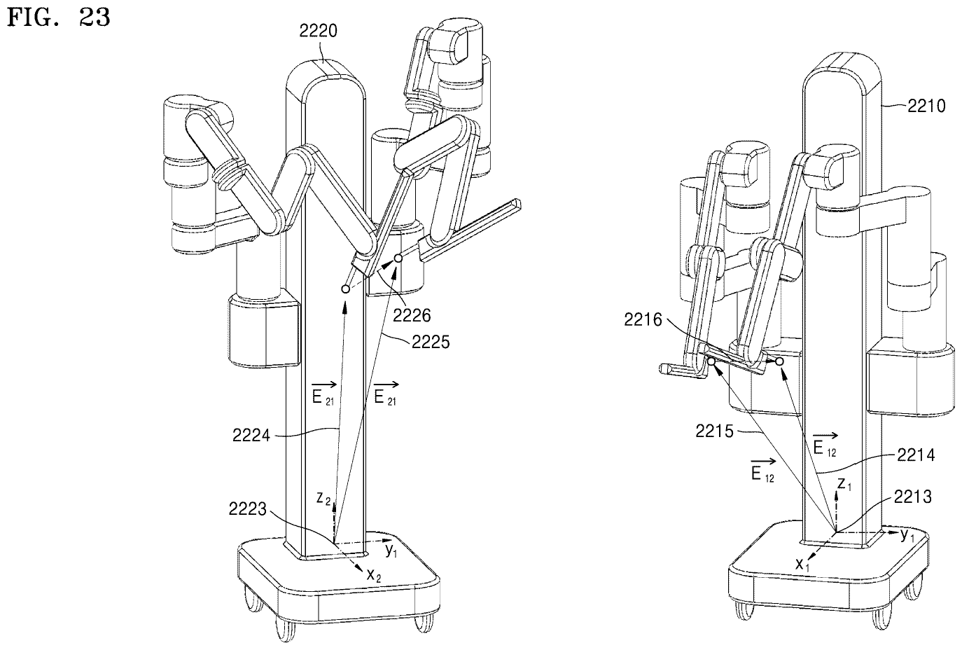

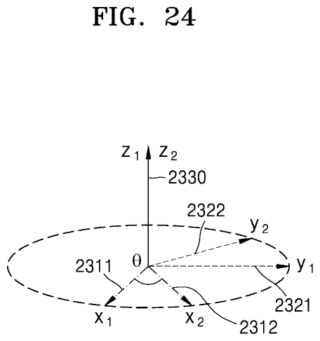

FIGS. 22A and 22B are views for describing an example of the first robot and the second robot according to an embodiment. FIG. 23 is a view for describing an example of generating first position information and second position information using positions of the first robot and the second robot, respectively, illustrated in FIGS. 22A and 22B. FIG. 24 is a diagram for describing an example of generating third position information by the processor according to an embodiment.

Referring to FIG. 22A, a 1-1 end effector, which is a first-1 component of a first robot 2210, may be located at a first position reference point 2211. In some embodiments, a 1-2 end effector, which is a first-2 component of the first robot 2210, may be located at a second position reference point 2212. In some embodiments, a 2-1 end effector, which is a second-1 component of a second robot 2220, may be located at a first position reference point 2222. In some embodiments, a 2-2 end effector, which is a second-2 component of the second robot 2220, may be located at a second position reference point 2221. The first position reference point 2211 and the first position reference point 2222 are illustrated separately for convenience of description, but refer to the same position. In some embodiments, the second position reference point 2212 and the second position reference point 2221 are illustrated separately for convenience of description, but refer to the same position.

A relative position from a position 2213 of the first robot to each of the 1-1 end effector and the 1-2 end effector may be determined based on preset lengths of robot links, as well as angle sensors and position sensors mounted on robot joints. Meanwhile, the end effector is not limited to the end of the robot, and may be any position at which the robot may securely fix its position. This is equally applicable to a position 2223 of the second robot, the 2-1 end effector and the 2-2 end effector.

x1, y1, and z1 each refer to a unit vector set based on the position 2213 of the first robot 2210. x2, y2, and z2 each refer to a unit vector set based on the position 2223 of the second robot 2220. Each of the unit vectors may be set with the center of a body of each of the first and second robots as the origin.

The processor 2011 may calculate vector values for the first position reference point 2211 and the second position reference point 2212 based on x1, y1, and z1. In some embodiments, the processor 2011 may calculate a first-1 intermediate vector value 2215 based on the first position reference point 2211 and the position 2213 of the first robot 2210. In some embodiments, the processor 2011 may calculate a first-2 intermediate vector value 2214 based on the second position reference point 2212 and the position 2213 of the first robot 2210. In some embodiments, the processor 2011 may calculate a first vector value 2216 based on the first-1 intermediate vector value 2215 and the first-2 intermediate vector value 2214.

In some embodiments, the processor 2011 may calculate vector values for the first position reference point 2222 and the second position reference point 2221 based on x2, y2, and z2. In some embodiments, the processor 2011 may calculate a second-1 intermediate vector value 2225 based on the first position reference point 2222 and the position 2223 of the second robot 2220. In some embodiments, the processor 2011 may calculate a second-2 intermediate vector value 2224 based on the second position reference point 2221 and the position 2223 of the second robot 2220. In some embodiments, the processor 2011 may calculate a second vector value 2226 based on the second-1 intermediate vector value 2225 and the second-2 intermediate vector value 2224.

Meanwhile, the processor 2011 may generate third position information regarding a relationship between the position of the first robot and the position of the second robot by using a correlation between the calculated first and second vector values 2216 and 2226.

The processor 2011 may use a direction vector (a, b, c) of the first vector value 2216 and a direction vector (d, e, f) of the second vector value 2226 to derive Equation 1 below.

$$a \vec{x 1} + b \vec{y 1} + c \vec{z 1} = d \vec{x 2} + e \vec{y 2} + f \vec{z 2} \qquad [ Equation 1 ]$$

The first vector value 2216 and the second vector value 2226 may have a correlation as shown in Equation 1. Based on this, the processor 2011 may calculate a relative angle θ using Equation 2 below.

$$( a \vec{x 1} + b \vec{y 1} + c \vec{z 1} ) \cdot ( d \vec{x 2} + e \vec{y 2} + f \vec{z 2} ) = 1 \qquad [ Equation 2 ]$$

Referring to Equation 2, the processor 2011 may calculate a relative angle θ between x1 (2311) and x2 (2312) by using the fact that the dot product of identical unit vectors has a value of 1. Here, z1 (2330) and z2 (2330) correspond to the same unit vector, and thus the dot product of z1 (2330) and z2 (2330) has a value of 1.

In other words, the processor 2011 may calculate the relative angle θ based on the direction vector (a, b, c) of the first vector value 2216, the direction vector (d, e, f) of the second vector value 2226, the unit vectors x1 (2311), y1 (2321), and z1 (2330) defined with respect to the position 2213 of the first robot 2210, and the unit vectors x2 (2312), y2 (2322), and z2 (2330) defined with respect to the position 2223 of the second robot 2220. Here, the relative angle θ may be included in the third position information.

Meanwhile, the processor 2011 may generate driving information based on the third position information. In some embodiments, the processor 2011 may control at least one of the first robot and the second robot based on the driving information. For example, the processor 2011 may control the surgical instrument mounted on the second robot based on the driving information that is generated based on the third position information. At this time, a direction of motion of the surgical instrument may align with a direction of motion of the surgical instrument shown in the image captured by the camera mounted on the first robot.

$${\vec{p}}_{sTool} = {R}_{sTool \to sRobot} {R}_{sRobot \to cRobot} {R}_{cRobot \to cView} {\vec{p}}_{master} \qquad [ Equation 3 ]$$

Referring to Equation 3, the processor 2011 may calculate driving information \vec{p}_{sTool} based on operation information \vec{p}ₘₐₛₜₑᵣ of the master robot, transformation information R_{cRobot→cView} between the coordinates of the image captured by the camera and the coordinates of the first robot, transformation information R_{sRobot→cRobot} between the position of the first robot and the position of the second robot, and transformation information (R_{sTool→sRobot}) between the position of the surgical instrument and the position of the second robot.

The operation information \vec{p}ₘₐₛₜₑᵣ of the master robot may include the operation information of the master robot generated by the user's manipulation, and may be the same as operation information \vec{p}_{cView} of the surgical instrument captured by the camera mounted on the first robot.

The transformation information R_{cRobot→cView} between the coordinates of the image captured by the camera and the coordinates of the first robot may include a transformation matrix between the coordinates of the image captured by the camera and the coordinates of the first robot. The transformation information R_{cRobot→cView} between the coordinates of the image captured by the camera and the coordinates of the first robot may be determined based on kinematic information of each of the first and second robots (e.g., the position of the first robot, the position of the second robot, and the like).

The transformation information R_{sRobot→cRobot} between the position of the first robot and the position of the second robot may include a transformation matrix of the position of the first robot and the position of the second robot. Here, the transformation information R_{sRobot→cRobot} of the position of the first robot and the position of the second robot may be determined based on the relative angle θ. In other words, the transformation information R_{sRobot→cRobot} between the position of the first robot and the position of the second robot may be determined by the relative angle θ between the first robot, on which the camera is mounted, and the second robot, on which the surgical instrument is mounted, around an axis perpendicular to the ground.

The transformation information R_{sTool→sRobot} between the position of the surgical instrument and the position of the second robot may include a transformation matrix of the position of the surgical instrument and the position of the second robot. The transformation information R_{sTool→sRobot}
between the position of the surgical instrument and the position of the second robot may be determined based on the kinematic information of each of the first and second robots (e.g., the position of the first robot, the position of the second robot, and the like).

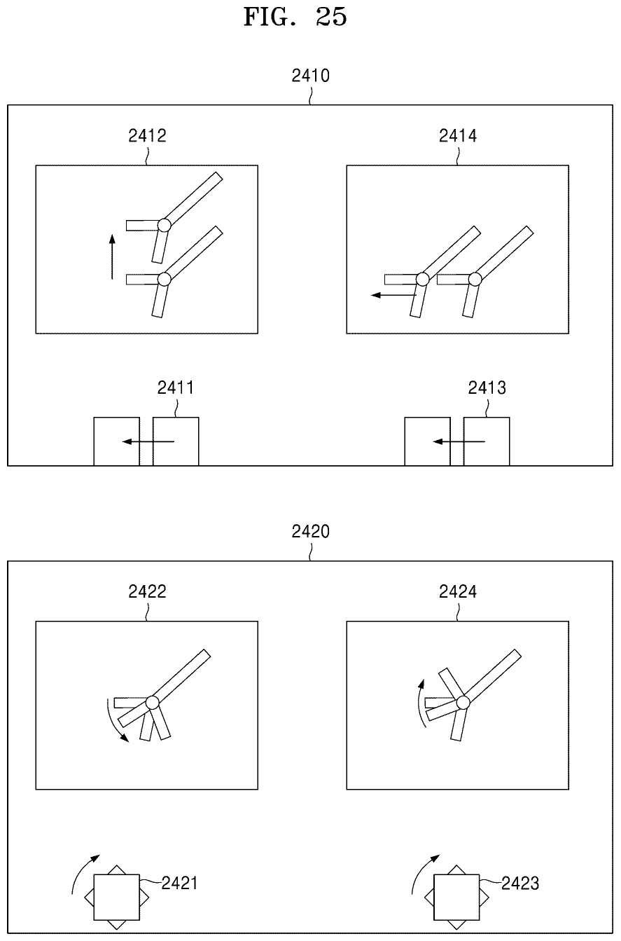

FIG. 25 is a view for describing an example of the surgical instrument operating based on the driving information according to an embodiment.

Referring to FIG. 25, display members 2412, 2414, 2422, and 2424 may display images captured by the camera mounted on the first robot. The camera mounted on the first robot may capture images of the surgical instrument mounted on the second robot.

First, in relation to a manipulation 2410 for moving the surgical instrument, when the member 2411, which allows a user to manipulate the position and function of the surgical instrument, is moved to the left, but the surgical instrument is shown on the display member 2412 as moving vertically, it becomes difficult for the user to intuitively manipulate the surgical instrument.

Meanwhile, in relation to the manipulation 2410 for moving the surgical instrument, when a member 2413, which allows a user to manipulate the position and function of the surgical instrument, is moved in a left and right direction, the surgical instrument, which operates based on the above-described driving information, is shown on the display member 2414 as moving to the left. Accordingly, the user can more accurately reflect his or her intuitive manipulation and perform surgery through the surgical robot.

In some embodiments, in relation to a manipulation 2420 for moving the surgical instrument, when a member 2421, which allows a user to manipulate the position and function of the surgical instrument, is moved in the clockwise direction, but the surgical instrument is shown on the display member 2422 as moving in the counterclockwise direction, it becomes difficult for the user to intuitively manipulate the surgical instrument.

Meanwhile, in relation to the manipulation 2420 for moving the surgical instrument, when a member 2423 that allows a user to manipulate the position and function of the surgical instrument is moved in the clockwise direction, the surgical instrument, which operates based on the above-described driving information, is shown on the display member 2424 as moving in the clockwise direction. Accordingly, the user can more accurately reflect his or her intuitive manipulation and perform surgery through the surgical robot.

As described above, the processor 2011 sets the first position reference point and the second position reference point, generates the first position information regarding the relationship among the first position reference point, the second position reference point, and the position of the first robot, generates the second position information regarding the relationship among the first position reference point, the second position reference point, and the position of the second robot, and generates the third position information regarding the relationship between the position of the first robot and the position of the second robot based on the first position information and the second position information, thereby allowing the surgical robot's operation to more accurately and intuitively reflect the user's manipulations.

According to the above-described technical solutions of the present disclosure, user manipulation can be more accurately and intuitively reflected in an operation of a surgical robot, by setting a first position reference point and a second position reference point, generating first position information regarding a relationship among the first position reference point, the second position reference point, and a position of a first robot, generating second position information regarding a relationship between the first position reference point, the second position reference point, and a position of a second robot, and generating third position information regarding a relationship among the position of the first robot and the position of the second robot based on the first position information and the second position information.

In some embodiments, in the present disclosure, user manipulation can be intuitively reflected in motions of a plurality of robotic arms included in each of the plurality of slave robots.

The effects of the present disclosure are not limited to those mentioned above, and other effects not mentioned may be clearly understood by those of ordinary skill in the art from the following description.

The above-described method may be recorded as a program that may be executed on a computer, and may be implemented in a general-purpose digital computer operating the program using a computer-readable recording medium. In some embodiments, the structure of the data used in the method described above may be recorded on a computer-readable recording medium through various means. Examples of the computer-readable recording medium include storage media such as magnetic storage media (e.g., ROM, floppy disks, hard disks, and the like), and optical read media (e.g., CD-ROMs, DVDs, and the like).

Meanwhile, the above-described method may be included and provided in a computer program product. The computer program product may be traded as a product between a seller and a buyer. The computer program product may be distributed in the form of a machine-readable storage medium (e.g., compact disc read-only memory (CD-ROM)), or be distributed (e.g., downloaded or uploaded) online via an application store (e.g., PlayStore™), or between two user devices (e.g., smart phones) directly. When distributed online, at least a part of the computer program product may be temporarily generated or at least temporarily stored in the machine-readable storage medium, such as memory of the manufacturer's server, a server of the application store, or a relay server.

It will be understood by those skilled in the art to which the present embodiment pertains that the present disclosure may be implemented in modified forms without departing from the spirit and scope of the present disclosure. Therefore, the disclosed methods are should be considered in an illustrative aspect rather than a restrictive aspect. The scope of the present disclosure should be defined by the claims rather than the above-mentioned description, and equivalents to the claims should be interpreted to fall within the present disclosure.

What is claimed is:

1. A method comprising:
setting a first position reference point and a second position reference point;
generating first position information regarding a relationship among the first position reference point, the second position reference point, and a position of a first robot, and
generating second position information regarding a relationship among the first position reference point, the second position reference point, and a position of a second robot; and
generating third position information regarding a relationship between the position of the first robot and the position of the second robot based on the first position information and the second position information.

2. The method of claim 1, wherein
the first position information includes relative position information of the first robot determined based on the first position reference point and the second position reference point, and
the second position information includes relative position information of the second robot determined based on the first position reference point and the second position reference point.

3. The method of claim 1, wherein
at the first position reference point,
a first-1 component of the first robot and a second-1 component of the second robot are located, and
at the second position reference point,
a first-2 component of the first robot and a second-2 component of the second robot are located.

4. The method of claim 1, wherein
the generating of the first position information and the second position information includes
calculating a first vector value for the first position reference point and the second position reference point based on the position of the first robot, and
calculating a second vector value for the first position reference point and the second position reference point based on the position of the second robot, and
the first position information includes
the first vector value, and
the second position information includes
the second vector value.

5. The method of claim 4, wherein
the calculating of the first vector value further includes:
calculating a first-1 intermediate vector value based on the first position reference point and the position of the first robot;
calculating a first-2 intermediate vector value based on the second position reference point and the position of the first robot; and
calculating the first vector value based on the first-1 intermediate vector value and the first-2 intermediate vector value, and
the calculating of the second vector value further includes:
calculating a second-1 intermediate vector value based on the first position reference point and the position of the second robot;
calculating a second-2 intermediate vector value based on the second position reference point and the position of the second robot; and
calculating the second vector value based on the second-1 intermediate vector value and the second-2 intermediate vector value.

6. The method of claim 1, wherein
the generating of the third position information further includes
generating the third position information regarding the relationship between the position of the first robot and the position of the second robot based on a correlation between the first position information and the second position information, and
the third position information includes
a relative angle generated based on the position of the first robot and the position of the second robot.

7. The method of claim 1, further comprising
controlling at least one of the first robot and the second robot based on the third position information.

8. The method of claim 7, wherein
the controlling further includes:
generating driving information based on an operation of the master robot, which controls the first robot and the second robot, and on a transformation relationship among the position of the first robot equipped with a camera, the position of the second robot equipped with a surgical instrument, and a position of the surgical instrument; and
controlling at least one of the first robot and the second robot based on the driving information.

9. The method of claim 8, wherein
the generating of the driving information further includes:
generating first intermediate driving information based on a transformation relationship between a position of an image captured by the camera and the position of the first robot, and operation information of the master robot;
generating second intermediate driving information based on a transformation relationship between the position of the first robot and the position of the second robot, and the first intermediate driving information; and
generating the driving information based on a transformation relationship between the position of the surgical instrument and the position of the second robot, and the second intermediate driving information.

10. An apparatus comprising:
at least one memory; and
at least one processor,
wherein the at least one processor is configured to:
set a first position reference point and a second position reference point;
generate first position information regarding a relationship among the first position reference point, the second position reference point, and a position of a first robot, and generate second position information regarding a relationship among the first position reference point, the second position reference point, and a position of a second robot; and
generate third position information regarding a relationship between the position of the first robot and the position of the second robot based on the first position information and the second position information.

11. The apparatus of claim 10, wherein
the first position information includes
relative position information of the first robot determined based on the first position reference point and the second position reference point, and
the second position information includes
relative position information of the second robot determined based on the first position reference point and the second position reference point.

12. The apparatus of claim 10, wherein
at the first position reference point,
a first-1 component of the first robot and a second-1 component of the second robot are located, and
at the second position reference point,
a first-2 component of the first robot and a second-2 component of the second robot are located.

13. The apparatus of claim 10, wherein
the at least one processor is further configured to
calculate a first vector value for the first position reference point and the second position reference point based on the position of the first robot, and
calculate a second vector value for the first position reference point and the second position reference point based on the position of the second robot, and
the first position information includes
the first vector value, and
the second position information includes
the second vector value.

14. The apparatus of claim 13, wherein
the at least one processor is further configured to:
calculate a first-1 intermediate vector value based on the first position reference point and the position of the first robot, calculate a first-2 intermediate vector value based on the second position reference point and the position of the first robot, and calculate the first vector value based on the first-1 intermediate vector value and the first-2 intermediate vector value; and
calculate a second-1 intermediate vector value based on the first position reference point and the position of the second robot, calculate a second-2 intermediate vector value based on the second position reference point and the position of the second robot, and calculate the second vector value based on the second-1 intermediate vector value and the second-2 intermediate vector value.

15. The apparatus of claim 10, wherein
the at least one processor is further configured to
generate the third position information regarding the relationship between the position of the first robot and the position of the second robot based on a correlation between the first position information and the second position information, and
the third position information includes
a relative angle generated based on the position of the first robot and the position of the second robot.

16. The apparatus of claim 10, wherein
the at least one processor is further configured to
control at least one of the first robot and the second robot based on the third position information.

17. The apparatus of claim 16, wherein
the at least one processor is further configured to:
generate driving information based on an operation of the master robot, which controls the first robot and the second robot, and on a transformation relationship among the position of the first robot equipped with a camera, the position of the second robot equipped with a surgical instrument, and a position of the surgical instrument; and
control at least one of the first robot and the second robot based on the driving information.

18. The apparatus of claim 17, wherein
the at least one processor is further configured to:
generate first intermediate driving information based on a transformation relationship between a position of an image captured by the camera and the position of the first robot, and operation information of the master robot;
generate second intermediate driving information based on a transformation relationship between the position of the first robot and the position of the second robot, and the first intermediate driving information; and
generate the driving information based on a transformation relationship between the position of the surgical instrument and the position of the second robot, and the second intermediate driving information.

19. A computer-readable recording medium having recorded thereon a program for causing a computer to execute the method of claim 1.
